(19) United States
(12) Patent Application Publication
(10) Pub. No.: US 2026/0318915 A1
(43) Pub. Date: Oct. 15, 2026

(54) ELISA kit for detecting Listeria monocytogenes and/or Listeria ivanovii infections, preparation method and use Thereof

(71) Applicant: Yangzhou University, Yangzhou (CN)

(72) Inventors: Xinan JIAO, Yangzhou (CN); Yao XU, Yangzhou (CN); Yuelan YIN, Yangzhou (CN); Fanzeng MENG, Yangzhou (CN); Ye WANG, Yangzhou (CN); Xiang CHEN, Yangzhou (CN); Dan GU, Yangzhou (CN)

(73) Assignee: Yangzhou University, Yangzhou (CN)

(21) Appl. No.: 19/119,111

(22) PCT Filed: Aug. 3, 2023
(86) PCT No.: PCT/CN2023/110937

§ 371 Date: Apr. 8, 2025

(30) Foreign Application Priority Data
Aug. 2, 2023 (CN) 202310966150.7

(51) Int. Cl. G01N 33/569; C12Q 1/28; G01N 33/68

(52) U.S. Cl. CPC G01N 33/56911; C12Q 1/28; G01N 33/6854; G01N 2333/195; G01N 2333/908; G01N 2469/20; G01N 2496/05

(57) ABSTRACT

The present disclosure discloses an indirect ELISA kit for detecting whether hosts are infected with Listeria monocytogenes and/or Listeria ivanovii. The coated antigens are the recombinant Listeria monocytogenes surface protein ActA, the outer membrane protein SmcL shared by serotype 4 h of Listeria monocytogenes and Listeria ivanovii, and the unique outer membrane protein I-inIH of Listeria ivanovii. An indirect ELISA method for each of these three antigens is established. By combining these three methods, the levels of ActA, SmcL and I-inIH antibodies in serum samples can be effectively monitored to detect listeriosis caused by Listeria monocytogenes and/or Listeria ivanovii. With high sensitivity, specificity, and stability, the kit provides an effective method for epidemiologic investigation and diagnosis of listeriosis.

### FIELD OF TECHNOLOGY

The present disclosure relates to the technical field of antibody detection, and in particular, to an ELISA kit for detecting antibodies against pathogenic Listeria monocytogenes in serum.

### BACKGROUND

Listeria monocytogenes (hereinafter referred to as Lm) and Listeria ivanovii (hereinafter referred to as Liv) are the main pathogenic species of the genus Listeria, which can cause listeriosis in humans and animals. Upon infection, Listeria monocytogenes can inflict substantial economic damage to livestock farms. Listeria monocytogenes carried by animals can enter the food supply chain through slaughter and processing stages. As these infected animal products undergo processing, transportation, and sales, they eventually reach the consumer's table, leading to potential human infections with a mortality rate as high as 30%. Listeriosis is mainly caused by serotype 1/2b and 4b strains of spectrum I and serotype 1/2a strain of spectrum II. For example, the highly virulent ST626 strain, isolated during a listeriosis outbreak in a sheep farm, belongs to the newly named serotype 4 h of Listeria monocytogenes. Listeria ivanovii mainly infects ruminants, for example, sheep herd is a susceptible host of Listeria in the natural environment. Sheep listeriosis brings about huge economic losses to the livestock industry. In recent years, there have been reports of cases of Listeria monocytogenes causing gastroenteritis and septicemia in humans, which warrant close attention.

Strengthening the screening for listeriosis in animal farming stages helps identify infections at an early stage and implement corresponding prevention and control measures, thereby preventing diseased livestock from entering the food supply chain, which is of great importance for public health safety. The “gold standard” for the detection of listeriosis involves the isolation of the pathogen. However, this method is time-consuming and often leads to inaccurate results due to the short survival time of live bacteria and intermittent shedding of bacteria. Serologic testing methods are commonly used to aid in the detection of infectious diseases, complementing and supporting traditional bacterial isolation and culture methods. The ELISA method has the advantages of easy operation, rapid results, and the ability to screen large quantities of samples in high throughput. It is widely used in epidemiological investigations and clinical immunological monitoring in sheep farms. Commercial kits for detecting listeriosis employ the indirect ELISA method based on Listerolys O (LLO), which are unable to differentiate between infections caused by Listeria monocytogenes and Listeria ivanovii and cross-react with serum antibodies from infections caused by other bacteria, such as streptococci.

ActA, an important virulence factor of Listeria monocytogenes, is located on the cell wall surface and has been confirmed to be highly immunogenicity. Both serotype 4 h of Listeria monocytogenes and Listeria ivanovii possess an outer membrane protein named SmcL, and a unique outer membrane protein of Listeria ivanovii is I-inIH.

It is urgent to develop a kit that can simultaneously detect Listeria monocytogenes and Listeria ivanovii and differentiate between infections caused by Listeria monocytogenes and Listeria ivanovii.

### SUMMARY

In the present disclosure, the surface protein ActA of Listeria monocytogenes, the outer membrane protein SmcL of serotype 4 h of Listeria monocytogenes and Listeria ivanovii, and the outer membrane protein I-inIH of Listeria ivanovii are selected to serve as detection antigens, so as to establish an indirect ELISA method and prepare a kit. The kit not only can detect whether the sample is infected with Listeria monocytogenes, but also can differentiate infections caused by Listeria monocytogenes and/or Listeria ivanovii, as well as infections caused by Listeria monocytogenes rather than serotype 4 h of Listeria monocytogenes.

The present disclosure provides an indirect ELISA kit for detecting Listeria monocytogenes and/or Listeria ivanovii. The kit comprises carriers coating ActA, SmcL, and I-inIH proteins, respectively, a standard positive control and a standard negative control, and detection reagents for detecting antigen-antibody reactions of ActA, SmcL, and I-inIH proteins with an antibody.

The present disclosure uses ActA, SmcL, and I-inIH antigens simultaneously for the first time to detect whether a host is infected with Listeria monocytogenes, Listeria ivanovii, or both Listeria monocytogenes and Listeria ivanovii. The kit of the present disclosure has high specificity and sensitivity. The inventors find that ActA, SmcL, and I-inIH proteins have good conservation and antigenicity, and employ them as coated antigens for the preparation of an indirect ELISA kit. In view of this, the inventors developed an indirect ELISA kit for differentiating infections caused by Listeria monocytogenes and Listeria ivanovii.

The present disclosure also provides a method for preparing an indirect ELISA kit, comprising the following steps: (1) coating: coating ActA, SmcL, and I-inIH proteins on ELISA plates, respectively, wherein an amino acid sequence of the ActA protein is shown as SEQ ID NO: 1, an amino acid sequence of the SmcL protein is shown as SEQ ID NO: 2, and an amino acid sequence of the I-inIH protein is shown as SEQ ID NO: 3; (2) blocking: using a blocking buffer to block the ELISA plates that have been coated with ActA, SmcL, and I-inIH proteins as described in step (1); (3) preparing a standard positive control and a standard negative control; (4) preparing an enzyme-labeled secondary antibody and a substrate solution. The ELISA plates prepared in the step (2), the standard positive control and the standard negative control prepared in the step (3), the enzyme-labeled secondary antibody and the substrate solution prepared in the step (4), are assembled to obtain the indirect ELISA kit.

The present disclosure optimizes the conditions for preparing the indirect ELISA kit, so as to allow the indirect ELISA kit highly specific and sensitive when detecting Listeria monocytogenes and Listeria ivanovii.

The present disclosure further provides a use of the indirect ELISA kit in the preparation of a detection reagent for detecting Listeria monocytogenes and/or Listeria ivanovii. The indirect ELISA kit of the present disclosure can differentiate infections caused by Listeria monocytogenes, Listeria ivanovii, or both Listeria monocytogenes and Listeria ivanovii. Additionally, the infections caused by serotype 4 h of Listeria monocytogenes or other serotypes of Listeria monocytogenes can also be differentiated.

As described above, the indirect ELISA kit for detecting Listeria monocytogenes and/or Listeria ivanovii, method, and use in the present disclosure have the following beneficial effects:
1) The recombinant protein I-inIH of Listeria ivanovii is cloned and expressed for the first time in the present disclosure. Immunoblotting shows that recombinant ActA, SmcL, and I-inIH proteins are all well reactive, specifically, serotype 4 h of Listeria monocytogenes XYSN infection induces the production of antibodies against ActA and SmcL proteins in Hu sheep, and Listeria ivanovii strain XYSL infected mice produce characteristic antibodies against I-inIH, suggesting that ActA, SmcL, and I-inIH proteins all have good immunogenicity.
2) The kit for detecting Listeria monocytogenes and/or Listeria ivanovii of the present disclosure and the method thereof can diagnose whether or not the host is suffering from listeriosis and the species of Listeria monocytogenes with which the host is infected by detecting corresponding antibodies. The kit can differentiate infections caused by Listeria monocytogenes, Listeria ivanovii, or both Listeria monocytogenes and Listeria ivanovii, and has high sensitivity, strong specificity, and good stability. The present disclosure can minimize the false positive rate and avoid the interference caused by other bacterial antigens belonging to Listeria. The present disclosure offers higher specificity than the indirect ELISA detection method based on LLO antigen, which is commonly used in veterinary clinics.
3) The method of the present disclosure addresses the lack of serological detection methods for differentiating infections caused by Listeria monocytogenes and Listeria ivanovii and can be used for epidemiological investigations of Listeria infection in host farms as well as for clinical immunological monitoring.

### BRIEF DESCRIPTION OF THE DRAWINGS

FIGS. 1-13 show exemplary embodiments of the present disclosure and are used in conjunction with their description to explain the principles of the present disclosure. These drawings are provided to contribute to a further understanding of the present disclosure, and are included as a part of this specification.

FIGS. 1A, 1B, and 1C are schematic diagrams of PCR amplification of ActA, SmcL, and I-inIH genes, respectively, wherein lane M represents DL2000 DNA molecular weight standard, lanes 1 and 2 represent ActA, SmcL, and I-inIH gene amplification products, respectively.

FIGS. 2A, 2B, and 2C are schematic diagrams for identifying double restriction enzyme digestion of recombinant plasmids inserted with an ActA, an SmcL, and an I-inIH gene, respectively, wherein lane M represents DL5000 DNA molecular weight standard, lane 1 represents double restriction enzyme digestion products of recombinant plasmids pET-30a-ActA, pET-30a-SmcL, and pET-30a-I-inIH, and lane 2 represents undigested recombinant plasmids pET-30a-ActA, pET-30a-SmcL, and pET-30a-I-inIH.

FIGS. 3A, 3B, and 3C are the schematic diagrams of induced expression of BL21(DE3)-pET-30a ActA, BL21(DE3)-pET-30a SmcL, and BL21(DE3)-pET-30a I-inIH transformed bacteria, respectively, wherein, lane M represents low molecular weight protein standard, and lane 1 represents purified rHis-ActA fusion protein, rHis-SmcL fusion protein, and rHis-I-inIH fusion protein.

FIGS. 4A, 4B, and 4C are the schematic diagrams of Western blot analysis of recombinant ActA protein, recombinant SmcL protein, and recombinant I-inIH protein, respectively, wherein, lane M represents low molecular weight protein standard, and lane 1 represents purified recombinant rHis-ActA fusion protein, recombinant rHis-SmcL fusion protein, and recombinant rHis-I-inIH fusion protein.

FIGS. 5A, 5B, 5C, and 5D are schematic diagrams of in vitro biological reactivity characterization of ActA, SmcL, and I-inIH antigens, wherein lane M represents low molecular weight protein standard; lane 1 represents rGST-ActA in FIGS. 5A, 5B, 50, and 5D; lane 2 represents rHis-SmcL in FIGS. 5A and 5D, and represents rGST-SmcL in FIGS. 5B and 50; and lane 3 represents rHis-I-inIH in FIGS. 5C and 5D, and represents rGST-I-inIH in FIGS. 5A and 5B.

FIGS. 6A, 6B, and 6C are diagrams showing the effects of different antigen coating concentrations and different enzyme-labeled secondary antibody dilutions on the OD₄₅₀ values of positive serum in the indirect ELISA methods for ActA, SmcL, and I-inIH, respectively.

FIGS. 7A, 7B, and 7C are diagrams showing the effect of different coating conditions on P/N values in the indirect ELISA method for ActA, SmcL, and I-inIH, respectively.

FIGS. 8A, 8B, and 8C are diagrams showing the effect of different blocking buffers on P/N values in the indirect ELISA method for ActA, SmcL, and I-inIH, respectively.

FIGS. 9A, 9B, and 9C are diagrams showing the effect of different blocking times on P/N values in the indirect ELISA method for ActA, SmcL, and I-inIH, respectively.

FIGS. 10A, 10B, and 10C are diagrams showing the effect of different serum primary antibody incubation times on P/N values in the indirect ELISA method for ActA, SmcL, and I-inIH, respectively.

FIGS. 11A, 11B, and 11C are diagrams showing the effect of different enzyme-labeled secondary antibody reaction times on P/N values in the indirect ELISA method for ActA, SmcL, and I-inIH, respectively.

FIGS. 12A, 12B, and 12C are diagrams showing the effect of different chromogenic times on P/N values in the indirect ELISA method for ActA, SmcL, and I-inIH, respectively.

FIGS. 13A, 13B, and 13C are the schematic diagrams showing changes of serum IgG antibody over time, after Hu sheep were infected by serotype 4 h of Listeria monocytogenes XYSN and mice were infected by Listeria ivanovii XYSL, in the indirect ELISA method for ActA, SmcL, and I-inIH, respectively.

### DETAILED DESCRIPTION

The present disclosure provides an indirect ELISA kit for detecting Listeria monocytogenes and/or Listeria ivanovii. The kit comprises carriers coating an ActA, an SmcL, and an I-inIH protein, respectively, a standard positive control and a standard negative control, and detection reagents for detecting antigen-antibody reactions of the ActA, SmcL, and I-inIH proteins with an antibody. Listeria monocytogenes is a Gram-positive small rod-shaped bacterium with no spores or capsules, and can be classified into 11 serotypes based on the somatic and flagellar antigens. It was first isolated from naturally infected rabbits in 1926, and was named Listeria monocytogenes because of the increase of monocytes in diseased animals. It is the main pathogenic bacterium causing diseases in both animals and humans.

Listeria ivanovii is another kind of pathogenic Listeria. Listeria ivanovii only causes diseases in animals and mainly infects ruminants. Sheep herd is a susceptible host of Listeria in the natural environment, and sheep listeriosis has brought about huge economic losses to the livestock industry.

ActA protein, an important virulence factor of Listeria monocytogenes, is located on the cell wall surface and has been confirmed to be highly immunogenicity.

Both serotype 4 h of Listeria monocytogenes and Listeria ivanovii possess an outer membrane protein named SmcL.

A unique outer membrane protein of Listeria ivanovii is I-inIH.

The mechanism of the ELISA detection process of the present disclosure is as follows. ELISA is based on the immobilization of antigens or antibodies and enzyme-labeled antigens or antibodies. Antigens or antibodies immobilized on the solid-phase carriers retain their immunological activity, and enzyme-labeled antibodies retain both their immunological activity and enzyme activity. During the detection, the to-be-tested samples (in which the antibody is to be tested) react with the antigens immobilized on the solid-phase carrier to form antigen-antibody complexes. These antigen-antibody complexes on the solid phase carrier are separated from other substances in the liquid by washing. The enzyme-labeled antibodies are then added and bind to the solid-phase carriers through reactions. The amount of enzyme on the solid-phase carrier is proportional to the amount of the target substance in the to-be-tested samples. After the substrate for the enzyme reaction is added, the substrate is catalyzed by the enzyme to form a colored product, and the amount of the product is directly related to the amount of the target substance in the to-be-tested samples. Therefore, qualitative or quantitative analysis can be carried out according to the intensity of the color developed.

The present disclosure confirms the immunogenicity of three proteins, including the ActA protein, SmcL protein, and I-inIH protein, and uses these three proteins for the detection of corresponding pathogenic infections for the first time. The combined use of these three proteins can differentiate the infections caused by Listeria monocytogenes and/or Listeria ivanovii, and differentiate whether the infection is caused by serotype 4 h of Listeria monocytogenes or other strains of Listeria monocytogenes.

In some embodiments, the amino acid sequence of the ActA protein is shown as SEQ ID NO: 1, the amino acid sequence of the SmcL protein is shown as SEQ ID NO: 2, and the amino acid sequence of the I-inIH protein is shown as SEQ ID NO: 3.

The amino acid sequence of the ActA protein has a homology of greater than or equal to 90% with the sequence shown as SEQ ID NO: 1. In some embodiments, the amino acid sequence of the ActA protein has a homology of 90%, 91%, 92%, 93%, 94%, 95%, 96%, 97%, 98%, 99%, or 100% with the sequence shown as SEQ ID NO: 1.

The amino acid sequence of the SmcL protein has a homology of greater than or equal to 90% with the sequence shown as SEQ ID NO: 2. In some embodiments, the amino acid sequence of the SmcL protein has a homology of 90%, 91%, 92%, 93%, 94%, 95%, 96%, 97%, 98%, 99%, or 100% with the sequence shown as SEQ ID NO: 2.

The amino acid sequence of the I-inIH protein has a homology of greater than or equal to 90% with the sequence shown as SEQ ID NO: 3. In some embodiments, the amino acid sequence of the I-inIH protein has a homology of 90%, 91%, 92%, 93%, 94%, 95%, 96%, 97%, 98%, 99%, or 100% with the sequence shown as SEQ ID NO: 3.

In one embodiment, the gene encoding sequences of the recombinant ActA, SmcL, and I-inIH proteins are shown as SEQ ID NO: 4, 5, and 6.

In some embodiments, the antibody comprises a monoclonal antibody or a polyclonal antibody against the ActA protein, a monoclonal antibody or a polyclonal antibody against the SmcL protein, and a monoclonal antibody or a polyclonal antibody against the I-inIH protein. The detection reagent comprises an enzyme-labeled secondary antibody and a substrate solution.

In some embodiments, the marker is selected from a group consisting of horseradish peroxidase (HRP), alkaline phosphatase, and biotin.

In some embodiments, the substrate solution is 3,3′,5,5′-tetramethylbenzidine (TMB).

In some embodiments, the indirect ELISA kit further comprises a sample dilution solution, a washing solution, a substrate solution, and a reaction stop solution.

In some embodiments, the sample dilution solution is a PBST solution containing 1% BSA, the washing solution is a PBST solution containing 0.05% of Tween 20 and with a pH of 7.2, and the reaction stop solution is a 2 mol/L H₂SO₄ solution.

In some embodiments, the standard positive control comprises positive serum from hosts infected with serotype 4 h of Listeria monocytogenes, other strains of Listeria monocytogenes, or Listeria ivanovii.

In some embodiments, the standard negative control comprises negative serum from hosts not infected with serotype 4 h of Listeria monocytogenes, other strains of Listeria monocytogenes, or Listeria ivanovii.

In some embodiments, carriers coated with the ActA protein, the SmcL protein, and the I-inIH protein use ELISA plates.

The present disclosure also provides a method for preparing the indirect ELISA kit, comprising the following steps:
(1) coating: coating the ActA protein, the SmcL protein, and the I-inIH protein on ELISA plates, respectively, wherein an amino acid sequence of the ActA protein is shown as SEQ ID NO: 1, an amino acid sequence of the SmcL protein is shown as SEQ ID NO: 2, and an amino acid sequence of the I-inIH protein is shown as SEQ ID NO: 3;
(2) blocking: using a blocking buffer to block the ELISA plates that have been coated with the ActA protein, the SmcL protein, and the I-inIH protein, respectively, as described in step (1);
(3) preparing the standard positive control and the standard negative control;
(4) preparing an enzyme-labeled secondary antibody and a substrate solution.
   Assembling the ELISA plates prepared in the step (2), the standard positive control and the standard negative control prepared in the step (3), the secondary antibody labeled with the marker and the substrate solution prepared in the step (4) to obtain the indirect ELISA kit.

In some embodiments, the method further comprises: in the step (1), dissolving the ActA protein, the SmcL protein, and the I-inIH protein in a CBS coating solution, respectively, and coating the ActA protein, the SmcL protein, and the I-inIH protein on the ELISA plates at a final concentration of 0.625, 0.625, and 1.25 g/ml, respectively. The ELISA plate to be coated with the ActA protein is placed at 37° C. for 2 h for the coating, and the ELISA plates to be coated with the SmcL protein and the I-inIH protein are placed at 4° C. for 16 h for the coating. In some embodiments, the indirect ELISA kit contains one ELISA plate coated with the ActA protein, the SmcL protein, and the I-inIH protein, respectively.

In some embodiments, the antigen may be pre-coated on an ELISA plate. In some embodiments, a blank ELISA plate and the antigens are provided, and the antigens can be coated on the blank ELISA plate by the operator before the detection.

In some embodiments, the antigen-coating solution includes a 0.05 mol/L carbonate (CBS) buffer containing the ActA, SmcL, or I-inIH protein with a pH of 9.6, respectively.

In some embodiments, the method further comprises: in the step (2), blocking the ELISA plate coated with the ActA protein with a PBS containing 5% of BSA for 2.5 h, and blocking the ELISA plates coated with the SmcL protein and the I-inIH protein with a PBS containing 5% of skimmed milk for 1.5 h.

In some embodiments, the method further comprises preparing a sample dilution solution, a washing solution, a substrate solution, and a reaction stop solution.

In some embodiments, the marker is selected from a group consisting of horseradish peroxidase (HRP), alkaline phosphatase, and biotin.

In some embodiments, the enzyme-labeled secondary antibody is horseradish peroxidase-labeled rabbit anti-sheep IgG antibody.

In some embodiments, the sample dilution solution is a PBST solution containing 1% w/v of BSA.

In some embodiments, the washing solution is a PBST solution containing 0.05% w/v of Tween 20 and with a pH of 7.2.

In some embodiments, the substrate solution is 3,3′,5,5′-tetramethylbenzidine (TMB).

In some embodiments, the reaction stop solution is a 2 mol/L H₂SO₄ solution.

In some embodiments, for the ActA protein, a coating concentration is 0.625 μg/mL, a coating temperature is 37° C., a coating time is 2 h, a blocking buffer is a PBS solution containing 5% of BSA, a blocking time is 2.5 h, and a dilution ration of the enzyme-labeled secondary antibody is 1:40,000.

In some embodiments, for the SmcL protein, a coating concentration is 0.625 μg/mL, a coating temperature is 4° C., a coating time is 16 h, a blocking buffer is a PBS solution containing 5% of skim milk powder, a blocking time is 1.5 h, and a dilution ration of the enzyme-labeled secondary antibody is 1:10,000.

The present disclosure also provides a use of the indirect ELISA kit in the preparation of a detection reagent for detecting Listeria monocytogenes and/or Listeria ivanovii.

In some embodiments, the detection reagent is highly specific for differentiating Listeria monocytogenes and Listeria ivanovii.

In some embodiments, the detection reagent is highly specific for differentiating serotype 4 h of Listeria monocytogenes and other strains of Listeria monocytogenes.

The present disclosure also provides a method for detecting Listeria monocytogenes and/or Listeria ivanovii using the indirect ELISA kit, including the following steps:
(1) incubation of to-be-tested samples: diluting with a sample dilution solution, followed by incubating the to-be-tested samples on the carriers coating the ActA protein, the SmcL protein, and the I-inIH protein, and adding the corresponding standard positive control and standard negative control;
(2) incubation of a secondary antibody: adding the secondary antibody to the carriers coating the ActA protein, the SmcL protein, and the I-inIH protein after the step (1) and performing incubation;
(3) coloration: adding a detection reagent to the carriers coating the ActA protein, the SmcL protein, and the I-inIH protein after the step (2), performing color development, then stopping the reaction with a reaction stop solution, and reading absorbance values at OD₄₅₀ nm;
(4) determination of results: calculating S/P ratios, wherein a calculation formula of S/P is S/P=(sample_{absorbance value at OD450 nm}−negative control_{mean absorbance value at OD450 nm})/(positive control_{mean absorbance value at OD450 nm}−negative control_{mean absorbance value at OD450 nm}). For the ActA protein, when a calculated S/P ratio is greater than 0.104, the to-be-tested samples are positive, and otherwise negative. For the SmcL protein, when a calculated S/P ratio is greater than 0.110, the to-be-tested samples are positive, and otherwise negative. For the I-inIH protein, when a calculated S/P ratio is greater than 0.137, the to-be-tested samples are positive, and otherwise negative. In some embodiments, the ActA, SmcL, and I-inIH proteins are recombinant proteins, the method for preparing the ActA, SmcL, and I-inIH recombinant proteins includes the following steps:
(a) performing PCR amplification using the genome of Listeria monocytogenes (LM) strain as a template to clone the truncated ActA gene fragment; and performing PCR amplification using the genome of Listeria ivanovii XYSL strain as a template to clone the truncated SmcL and I-inIH gene fragments.
(b) Separating the nucleic acid fragments of the recombinant proteins by electrophoresis, after gel recovering and purifying, ligating them with the Escherichia coli prokaryotic expression vector pET-30a to obtain the recombinant plasmids pET-30a-actA, pET-30a-smcL, and pET-30a-i-inIH, transforming DH5α competent cells to obtain prokaryotic recombinant strains, extracting the plasmids and performing double enzyme digestion electrophoresis for detection, and conducting sequencing analysis on the recombinant plasmids that are correctly identified by electrophoresis.
(c) Transforming the recombinant plasmids into Escherichia coli BL21(DE3) competent cells for prokaryotic expression to obtain three recombinant proteins, namely Listeria monocytogenes rHis-ActA protein and Listeria ivanovii rHis-SmcL and rHis-I-inIH proteins.
(d) Ultrasonically disrupting the bacterial culture of the prokaryotic expression, centrifuging to obtain the supernatant containing the target protein, purifying the supernatant using a His-tag fusion protein purification column for affinity chromatography, and collecting the elution after column purification to obtain the purified ActA, SmcL, and I-inIH recombinant proteins.

In some embodiments, the sequences of primer pair employed for the amplification in the step (a) are as shown as SEQ ID NO: 7, 8, 9, 10, 11, and 12.

In the ELISA detection of the to-be-tested serum samples, for the ActA protein, in some embodiments, a coating concentration is 0.625 μg/mL, a coating temperature is 37° C., a coating time is 2 h, a blocking buffer is a PBS solution containing 5% of BSA, a blocking time is 2.5 h, an incubation time of the serum is 1 h, and a dilution ration of the enzyme-labeled secondary antibody is 1:40,000, an incubation time for the enzyme-labeled secondary antibody is 30 min, and a time for the color development is 5 min. Under the above conditions, for the ActA protein, when a calculated S/P ratio is greater than 0.104, the to-be-tested serum samples are positive, and otherwise negative, wherein the S/P ratio is calculated according to an equation of S/P=(S−N)/(P−N).

In the ELISA detection of the to-be-tested serum samples, for the SmcL protein, in some embodiments, a coating concentration is 0.625 μg/mL, a coating temperature is 4° C., a coating time is 16 h, a blocking buffer is a PBS solution containing 5% of skim milk powder, a blocking time is 1.5 h, an incubation time of the serum is 2.5 h, and a dilution ration of the enzyme-labeled secondary antibody is 1:10,000, an incubation time for the enzyme-labeled secondary antibody is 60 min, and a time for the color development is 10 min. Under the above conditions, for the SmcL protein, when a calculated S/P ratio is greater than 0.110, the to-be-tested serum samples are positive, and otherwise negative, wherein the S/P ratio is calculated according to an equation of S/P=(S−N)/(P−N).

In the ELISA detection of the to-be-tested serum samples, for the I-inIH protein, in some embodiments, a coating concentration is 1.25 μg/mL, a coating temperature is 4° C., a coating time is 16 h, a blocking buffer is a PBS solution containing 5% of skim milk powder, a blocking time is 1.5 h, an incubation time of the serum is 2.5 h, and a dilution ration of the enzyme-labeled secondary antibody is 1:20,000, an incubation time for the enzyme-labeled secondary antibody is 60 min, and a time for the color development is 10 min. Under the above conditions, for the I-inIH protein, when a calculated S/P ratio is greater than 0.137, the to-be-tested serum samples are positive, and otherwise negative, wherein the S/P ratio is calculated according to an equation of S/P=(S−N)/(P−N).

In some embodiments, the method further comprises: in the step (1), incubating the to-be-tested samples at 37° C. for 1 h on the carrier coating the ActA protein, and incubating the to-be-tested samples at 37° C. for 2.5 h on the carrier coating the SmcL protein and the carrier coating the I-inIH protein, respectively.

In some embodiments, the to-be-tested samples in the step (1) are sera.

In some embodiments, the method further comprises: in the step (2), adding 100 μl of the secondary antibody with a dilution ratio of 1:20,000 to the carrier coating the ActA protein and the carrier coating the SmcL protein after the step (1) and performing incubation for 60 min, and adding 100 μl of the secondary antibody with a dilution ratio of 1:10,000 to the carrier coating the I-inIH protein and performing incubation for 60 min.

In some embodiments, in the step (3), a time for the color development for the carrier coating the ActA protein is 5 min, and a time for the color development for the carrier coating the SmcL protein and the carrier coating the I-inIH protein is 10 min.

In some embodiments, the detection method further includes the following: when detection results for the ActA protein, the SmcL protein, and the I-inIH protein all show negative, it indicates that a host does not suffer from listeriosis; when a detection result for the ActA protein shows positive and detection results for the SmcL protein and the I-inIH protein show negative, it indicates that the host is infected by Listeria monocytogenes rather than serotype 4 h of Listeria monocytogenes; when detection results for the ActA protein and the SmcL protein show positive and a detection result for the I-inIH protein shows negative, it indicates that the host is infected by serotype 4 h of Listeria monocytogenes; when a detection result for the ActA protein shows negative and detection results for the SmcL protein and the I-inIH protein show positive, it indicates that the host is infected by Listeria ivanovii; when detection results for the ActA protein, the SmcL protein, and the I-inIH protein all show positive, it indicates that the host is infected by both Listeria monocytogenes and Listeria ivanovii.

Detection results other than those mentioned above are regarded as invalid, and the reference of results from third-party diagnostic methods is needed.

The present disclosure also provides a use of an ActA protein, an SmcL protein, and an I-inIH protein in the preparation of an immunological detection reagent for detecting Listeria monocytogenes and/or Listeria ivanovii. The immunological detection reagent further includes a standard positive control and a standard negative control, and a detection reagent for detecting an antigen-antibody reaction of the ActA protein, the SmcL protein, and the I-inIH protein with an antibody.

In some embodiments, the immunological detection reagent is for differentiating Listeria monocytogenes and Listeria ivanovii.

In some embodiments, the immunological detection reagent is for differentiating serotype 4 h of Listeria monocytogenes and other strains of Listeria monocytogenes.

The present disclosure is further described below with reference to specific embodiments. It should be understood that the embodiments are just for describing the present disclosure instead of limiting the scope of the present disclosure. Those skilled in the art can easily understand other advantages and effects of the present disclosure according to the contents disclosed by the specification. The present disclosure may also be implemented or applied through other different specific implementation modes. Various modifications or changes may be made to all details in the specification based on different points of view and applications without departing from the spirit of the present disclosure.

It is also to be understood that the term of the present disclosure is used to describe the specific embodiments, instead of limiting the scope of the present disclosure. In the present specification and claims, the singular forms “a”, “an” and “the” also include the plural forms, unless specifically stated otherwise. When the numerical ranges are given by the embodiments, it is to be understood that the two endpoints of each numerical range and any value between the two endpoints may be selected unless otherwise stated.

Unless otherwise defined, all technical and scientific terms used in the present disclosure have the same meaning as commonly understood by one skill in the art. In addition to the specific method, equipment, and material used in the embodiments, any method, equipment, and material in the existing technology similar or equivalent to the method, equipment, and material mentioned in the embodiments of the present disclosure, within the knowledge of those skilled in the art based on the prior art and the teachings of the present disclosure, may also be used to realize the present disclosure.

Unless otherwise specified, the experimental methods, detection methods, and preparation methods disclosed in the present disclosure all employ conventional techniques in the technical field and related fields.

### Embodiment 1 Amplification of Genes Encoding the ActA, SmcL, and I-inIH Proteins and Construction and Expression of Expression Vectors

### 1. Primer Design and Synthesis

Three specific primer pairs were designed and synthesized with reference to the GenBank-published gene sequences encoding the ActA, SmcL, and I-inIH proteins. The sequences of the forward primer and reverse primer for amplification of the ActA gene are shown as SEQ ID NO: 7 and 10. The sequences of the forward primer and reverse primer for amplification of the SmcL gene are shown as SEQ ID NO: 8 and 11. The sequences of forward primer and reverse primer for amplification of the I-inIH gene are shown as SEQ ID NO: 9 and 12. Double enzyme digestion sites BamH I and Hind III are provided in the forward primers and reverse primers, respectively. The primers are synthesized, purified, and sequenced by Beijing Tsingke Biotech Co., Ltd.

| | | Sequence ID |
|---|---|---|
| Name of sequence | Sequence | Number |
| Forward primer for | GCCATGGCTGATATCGGATCCATGGC | SEQ ID NO: 7 |
| amplification of the | GACAGATAGCGAAGATT | |
| ActA gene | | |
| Reverse primer for | CTCGAGTGCGGCCGCAAGCTTTTAATT | SEQ ID NO: 10 |
| amplification of the | ATTTTTTCTTAATTGAATAATTTTGA | |
| ActA gene | | |
| Forward primer for | GCCATGGCTGATATCGGATCCATGAGT | SEQ ID NO: 8 |
| amplification of the | GATGAGTATCCAGGTAATTTTAA | |
| SmcL gene | | |
| Reverse primer for | TCGAGTGCGGCCGCAAGCTTTTAGTTA | SEQ ID NO: 11 |
| amplification of the | TTATCAGTGAAACCAACTACAGG | |
| SmcL gene | | |
| Forward primer for | GCCATGGCTGATATCGGATCCATGATT | SEQ ID NO: 9 |
| amplification of the I- | CTACATTCGATACCTATAAATGAA | |
| inIH gene | | |
| Reverse primer for | CTCGAGTGCGGCCGCAAGCTTTCACA | SEQ ID NO: 12 |
| amplification of the I- | TAAGTAGAGATTTCTTAAGTTG | |
| inIH gene | | |

### 2. PCR Amplification is Performed Using the Above Primers

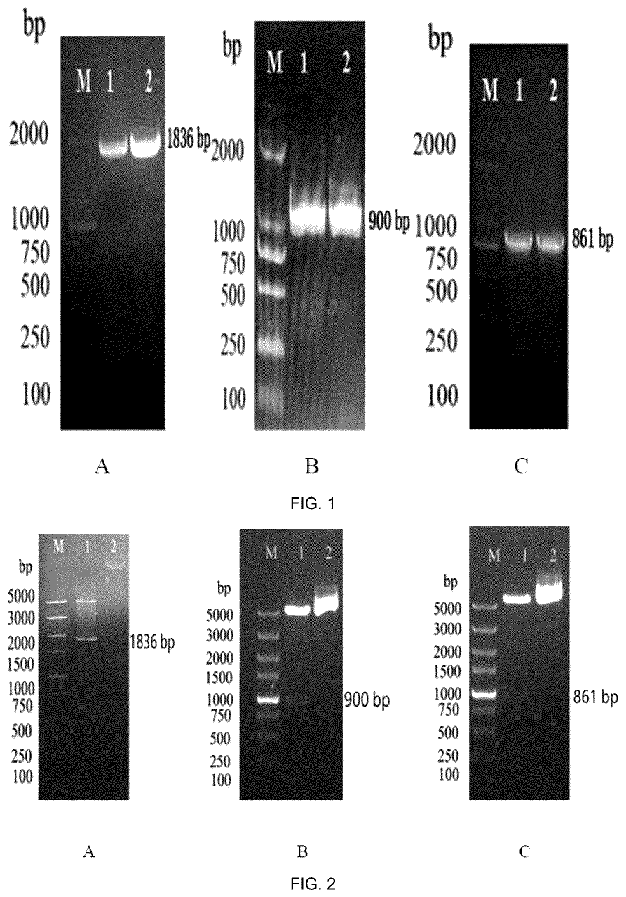

Listeria monocytogenes LM4 genome and Listeria ivanovii XYSL genome were employed as templates, the results of the PCR amplification are shown in FIG. 1. The PCR amplification reaction system is as follows: the total reaction volume is 50 μL, consisting of 2× Primestar max DNA Polymerase 25 μL, 2 μL of forward primer and reverse primer, respectively, 2 μL of template, and sterilized ddH₂O which makes the final volume up to 50 μL. PCR amplification reaction conditions are as follows: an initial denaturation at 98° C. for 5 minutes, followed by the cycling conditions: denaturation at 98° C. for 10 seconds, annealing at 55° C. for 15 seconds, and extension at 72° C. for 1 minute, with 34 cycles; and a final extension at 72° C. for 10 minutes.

### 3. Construction of Recombinant Plasmids

50 μL of PCR products were subjected to nucleic acid electrophoresis for identification. The products were separated by electrophoresis, gel-extracted, ligated to a linearized pET-30a vector, and then transformed into E. coli DH5α competent cells. The cells were plated on kanamycin-resistant (Kan+) plates and incubated overnight, white single colonies were picked for purification and identification. Plasmids extraction followed by double enzyme digestion electrophoresis was performed for verification, and the results are shown in FIG. 2. The plasmids that were correctly identified by electrophoresis were sent to Beijing Tsingke Biotech Co., Ltd for sequencing.

The amino acid sequence of the ActA protein is shown as SEQ ID NO: 1:

ATDSEDSSLNTDEWEEEKTEEQPSEVNTGPRYETAREVSSRDIKE
LEKSNKVRNTNKADLIAMLKEKAEKGPNINNNNSEQTENAAINEE
ASGADRPAIQVERRHPGLPSDSAAEIKKRRKAIASSDSELESLTY
PDKPTKVNKKKVAKESVADASESDLDSSMQSADESSPQPLKANQQ
PFFPKVFKKIKDAGKWVRDKIDENPEVKKAIVDKSAGLIDQLLTK
KKSEEVNASDFPPPPTDEELRLALPETPMLLGFNAPATSEPSSFE
FPPPPTDEELRLALPETPMLLGFNAPATSEPSSFEFPPPPTEDEL
EIIRETASSLDSSFTRGDLASLRNAINRHSQNFSDFPPIPTEEEL
NGRGGRPTSEEFSSLNSGDFTDDENSETTEEEIDRLADLRDRGTG
KHSRNAGFLPLNPFASSPVPSLSPKVSKISAPALISDITKKTPFK
NPSQPLNVFNKKTTTKTVTKKPTPVKTAPKLAELPATKPQETVLR
ENKTPFIEKQAETNKQSINMPSLPVIQKEATESDKEEMKPQTEEK
MVEESESANNANGKNRSAGIEEGKLIAKSAEDEKAKEEPGNHTTL
ILAMLAIGVFSLGAFIKIIQLRKNN

The amino acid sequence of the SmcL protein is shown as SEQ ID NO: 2:

SDEYPGNFKITSHNVYLFSRNIYPNWGQMHRADLIAQADYMKNND
VVILNEAFDTSASHRLLNNLREMYPHQTPVIGRSKHGWDKTEGNY
SNFALEDGGVAVVSQWPIVEKSQHIFQRGGGADRLSNKGFAYVKI
MKNGKPYHIIGTHTQADDSLISKDTSRAIRAEQMQEIQTFIAKKN
IPKDEIIFIGGDLNVNYGTDEYHDMLKLLNVSSPANFNGQMATWD
PTTNSMLKESYPKAAPEYLDYIFVENGHARPHSWHNKVLHTKSPQ
WSVKSWFKTYTYQDFSDHYPVVGFTDNN

The amino acid sequence of the I-inIH protein is shown as SEQ ID NO: 3:

ILHSIPINEIFQDYGLANAVKRQLGKQSVTEVVSQRELDEIQKLN
GNGCNIKSIKGVEYLTNLTKLYLSYNHISDISALASLSQLEVLSL
KQNKLKDINSLANLTKLRCLYVSNNQLRDLWALEGLKNLEYVDAE
EQTCTNNPVVYKPKVVIPNTIKDMSGKSVTPNFISDNGEYINTDV
IWELPTYTTEVSYRFKDILNIGKDYVLFSGVVIQPVELEAPAAEN
APSINQTLPAASLTEALKKGLRKKAVTDISPKSELDKANEFHDEN
RGSAMFMQLKKSLLM

The gene sequence of the ActA is shown in SEQ ID NO: 4:

GCGACAGATAGCGAAGATTCTAGTCTAAACACAGATGAATGGGAA
GAAGAAAAAACAGAAGAGCAACCAAGCGAGGTAAATACGGGACCA
AGATACGAAACTGCACGTGAAGTAAGTTCACGTGATATTAAAGAA
CTAGAAAAATCGAATAAAGTGAGAAATACGAACAAAGCAGACCTA
ATAGCAATGTTGAAAGAAAAAGCAGAAAAAGGTCCAAATATCAAT
AATAACAACAGTGAACAAACTGAGAATGCGGCTATAAATGAAGAG
GCTTCAGGAGCCGACCGACCAGCTATACAAGTGGAGCGTCGTCAT
CCAGGATTGCCATCGGATAGCGCAGCGGAAATTAAAAAAAGAAGG
AAAGCCATAGCATCATCGGATAGTGAGCTTGAAAGCCTTACTTAT
CCGGATAAACCAACAAAAGTAAATAAGAAAAAAGTGGCGAAAGAG
TCAGTTGCGGATGCTTCTGAAAGTGACTTAGATTCTAGCATGCAG
TCAGCAGATGAGTCTTCACCACAACCTTTAAAAGCAAACCAACAA
CCATTTTTCCCTAAAGTATTTAAAAAAATAAAAGATGCGGGGAAA
TGGGTACGTGATAAAATCGACGAAAATCCTGAAGTAAAGAAAGCG
ATTGTTGATAAAAGTGCAGGGTTAATTGACCAATTATTAACCAAA
AAGAAAAGTGAAGAGGTAAATGCTTCGGACTTCCCGCCACCACCT
ACGGATGAAGAGTTAAGACTTGCTTTGCCAGAGACACCAATGCTT
CTTGGTTTTAATGCTCCTGCTACATCAGAACCGAGCTCATTCGAA
TTTCCACCACCACCTACGGATGAAGAGTTAAGACTTGCTTTGCCA
GAGACGCCAATGCTTCTTGGTTTTAATGCTCCTGCTACATCGGAA
CCGAGCTCGTTCGAATTTCCACCGCCTCCAACAGAAGATGAACTA
GAAATCATCCGGGAAACAGCATCCTCGCTAGATTCTAGTTTTACA
AGAGGGGATTTAGCTAGTTTGAGAAATGCTATTAATCGCCATAGT
CAAAATTTCTCTGATTTCCCACCAATCCCAACAGAAGAAGAGTTG
AACGGGAGAGGGGGTAGACCAACATCTGAAGAATTTAGTTCGCTG
AATAGTGGTGATTTTACAGATGACGAAAACAGCGAGACAACAGAA
GAAGAAATTGATCGCCTAGCTGATTTAAGAGATAGAGGAACAGGA
AAACACTCAAGAAATGCGGGTTTTTTACCATTAAATCCGTTTGCT
AGCAGCCCGGTTCCTTCGTTAAGTCCAAAGGTATCGAAAATAAGC
GCACCGGCTCTGATAAGTGACATAACTAAAAAAACGCCATTTAAG
AATCCATCACAGCCATTAAATGTGTTTAATAAAAAAACTACAACG
AAAACAGTGACTAAAAAACCAACCCCTGTAAAGACCGCACCAAAG
CTAGCAGAACTTCCTGCCACAAAACCACAAGAAACCGTACTTAGG
GAAAATAAAACACCCTTTATAGAAAAACAAGCAGAAACAAACAAG
CAGTCAATTAATATGCCGAGCCTACCAGTAATCCAAAAAGAAGCT
ACAGAGAGCGATAAAGAGGAAATGAAACCACAAACCGAGGAAAAA
ATGGTAGAGGAAAGCGAATCAGCTAATAACGCAAACGGAAAAAAT
CGTTCTGCTGGCATTGAAGAAGGAAAACTAATTGCTAAAAGTGCA
GAAGACGAAAAAGCGAAGGAAGAACCAGGGAACCATACGACGTTA
ATTCTTGCAATGTTAGCTATTGGCGTGTTCTCTTTAGGGGCGTTT
ATCAAAATTATTCAATTAAGAAAAAATAATTAA

The gene sequence of the SmcL is shown as SEQ ID NO: 5:

AGTGATGAGTATCCAGGTAATTTTAAGATTACTTCACACAACGTT
TATTTATTTTCTAGAAATATATATCCTAATTGGGGACAAATGCAC
CGAGCTGATTTAATTGCACAAGCGGACTATATGAAAAATAATGAT
GTCGTCATTTTAAACGAAGCCTTTGATACAAGTGCTTCACATCGA
CTATTAAATAACCTTAGAGAAATGTATCCTCATCAAACACCCGTA
ATTGGGAGAAGTAAACATGGTTGGGATAAAACGGAAGGAAATTAT
TCCAATTTTGCTTTGGAAGATGGTGGAGTGGCTGTCGTTAGCCAG
TGGCCTATTGTCGAAAAAAGCCAACATATTTTTCAGCGCGGCGGT
GGCGCAGATCGGCTCAGTAATAAAGGATTCGCCTATGTTAAAATT
ATGAAAAACGGAAAACCTTATCATATAATTGGAACTCATACCCAA
GCGGATGACTCGCTTATTTCTAAAGATACTAGCCGGGCGATTCGT
GCAGAACAAATGCAAGAAATTCAAACATTTATTGCCAAGAAAAAT
ATCCCTAAAGATGAAATTATCTTTATTGGAGGCGATTTGAACGTA
AACTACGGTACAGATGAATATCATGATATGTTAAAGCTTTTAAAT
GTTAGCTCTCCGGCAAACTTTAACGGACAGATGGCTACTTGGGAT
CCAACGACTAATTCGATGTTAAAAGAAAGTTATCCTAAAGCAGCA
CCAGAATACCTTGATTATATTTTTGTTGAAAACGGTCATGCACGT
CCACATTCATGGCATAACAAAGTCTTGCATACCAAGTCACCACAG
TGGTCTGTGAAATCTTGGTTTAAAACTTATACGTATCAAGACTTT
TCAGATCATTATCCTGTAGTTGGTTTCACTGATAATAACTAA

The gene sequence of the I-inIH is shown as SEQ ID NO: 6:

ATTCTACATTCGATACCTATAAATGAAATTTTTCAGGATTATGGT
TTAGCGAATGCAGTGAAACGACAATTAGGAAAGCAAAGTGTTACA
GAGGTTGTTTCACAAAGAGAACTAGATGAAATACAAAAGTTAAAT
GGTAATGGTTGCAACATTAAATCTATTAAAGGGGTAGAATATCTT
ACTAATTTAACAAAGTTATATCTATCTTATAATCATATAAGTGAT
ATTAGCGCGCTTGCTTCTTTGAGTCAACTTGAGGTATTATCTTTA
AAGCAGAATAAGCTAAAAGACATCAATTCATTAGCTAACTTGACC
AAATTAAGATGTTTGTATGTGAGTAACAATCAATTAAGAGATTTA
TGGGCATTAGAAGGGCTGAAGAATTTAGAGTATGTAGATGCAGAA
GAACAAACCTGCACCAATAATCCTGTTGTATACAAACCAAAAGTG
GTTATTCCAAATACGATTAAAGACATGAGCGGAAAGTCAGTTACA
CCAAATTTTATTAGCGATAATGGAGAATATATAAATACGGATGTT
ATATGGGAGTTGCCGACATATACAACGGAAGTAAGCTATAGATTT
AAGGATATTCTAAACATTGGGAAAGACTACGTTCTATTTAGTGGT
GTAGTAATACAGCCAGTGGAATTAGAGGCACCTGCTGCTGAAAAT
GCACCCTCTATTAACCAAACTTTACCAGCAGCAAGTCTAACAGAA
GCACTAAAAAAAGGCTTAAGAAAAAAGGCAGTTACGGATATTAGT
CCAAAAAGTGAATTAGATAAGGCGAATGAATTTCACGATGAAAAT
AGAGGAAGTGCAATGTTTATGCAACTTAAGAAATCTCTACTTATG
TGA

### 4. Construction and Expression of Expression Vectors

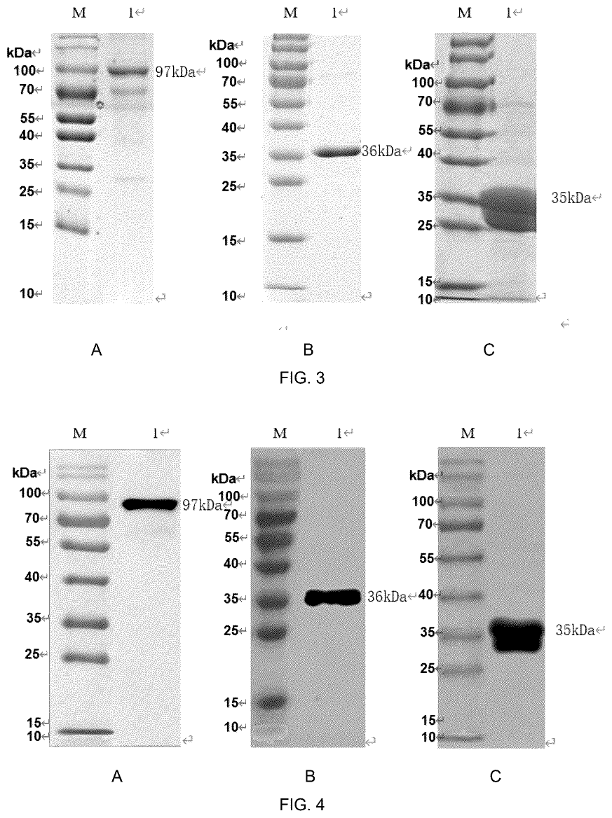

The positive recombinant plasmids were transformed into BL21(DE3) competent cells, and white colonies were picked for purification and identification. Single colonies of BL21(DE3) (pET-30a-ActA) identified as positive were inoculated into LB liquid medium containing Kan+ (100 μg/mL), and cultured with shaking at 37° C. until the OD value reached 0.4, then IPTG (0.2 mM/L) was added, and induction was carried out at 180 rpm, 37° C. for 5 h. The bacterial cells were collected by centrifugation. The bacterial cells were then lysed using an ultrasonic cell disruptor, and the supernatant after centrifugation was collected. The ActA protein was purified using an affinity chromatography column (His-Bind purification kit, purchased from Novagen). For rHis-SmcL and rHis-I-inIH, except for the induction temperature of 15° C. and the induction time of 20 h, the remaining steps were the same as above. Subsequently, SDS PAGE electrophoresis was performed, and the results are shown in FIG. 3, revealing that the target fusion protein fragments with molecular weights of 97 kDa, 36 kDa and 35 kDa are obtained.

### Embodiment 2 Western Blot and Bioreactivity Identification of the ActA, SmcL, and I-inIH Recombinant Proteins

### 1. Western Blot Identification of the ActA, SmcL, and I-inIH Proteins

After the purified recombinant fusion proteins were subjected to SDS PAGE electrophoresis, His monoclonal antibody solution was used as the primary antibody and HRP-labeled goat anti-mouse polyclonal antibody was used as the secondary antibody for Western blot. The reaction was developed with ECL enhanced chemiluminescence substrate, and the film was exposed in the dark for 2 minutes before reading. The results showed that the three Listeria recombinant proteins of the present disclosure can react with specific monoclonal antibodies, indicating that the proteins were correctly identified (FIG. 4). Purified and identified recombinant proteins were dialyzed for protein concentration determination.

### 2. Bioreactivity Identification of the ActA, SmcL, and I-inIH Proteins

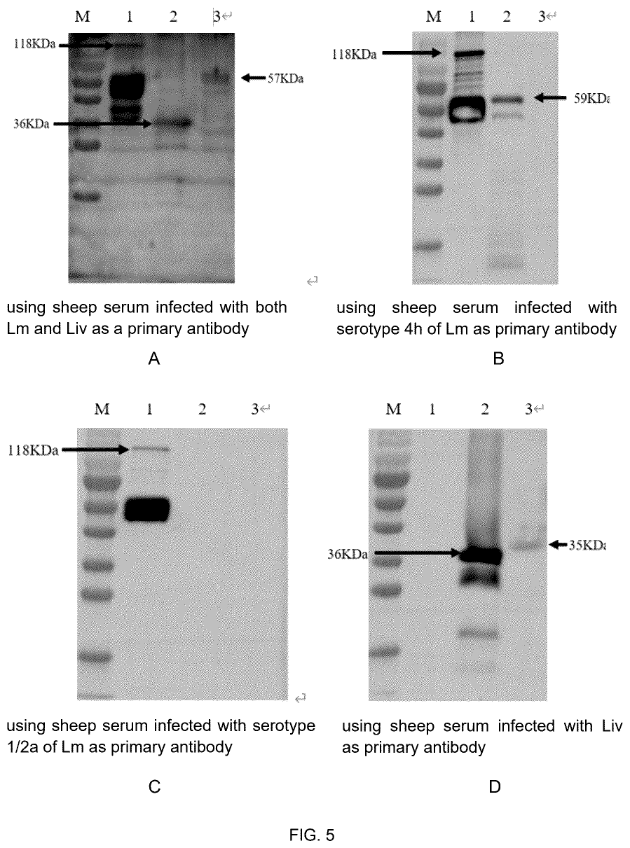

To verify the distribution of ActA, SmcL, and I-inIH proteins in serotype 4 h of Listeria monocytogenes, non-serotype 4 h of Listeria monocytogenes and Listeria ivanovii, the purified and identified three proteins were separately loaded in SDS-PAGE gel wells. After membrane transfer and blocking, a positive polyclonal goat serum infected with Listeria monocytogenes and/or Listeria ivanovii, respectively was used as the primary antibody for Western blot, and a rabbit anti-goat HRP-labeled antibody was used as the secondary antibody, followed by chemiluminescent development. Four different reaction conditions were set up: (1) sheep serum both infected with Listeria monocytogenes and Listeria ivanovii; (2) sheep serum infected with serotype 4 h of Listeria monocytogenes; (3) sheep serum infected with 1/2a of Listeria monocytogenes; (4) sheep serum infected with Listeria ivanovii. The specific distribution of ActA, SmcL, and I-inIH proteins in serotype 4 h of Listeria monocytogenes, non-serotype 4 h of Listeria monocytogenes and Listeria ivanovii is shown in Table 1 and FIG. 5.

TABLE 1
Distribution of ActA, SmcL and I-inIH antigens in serotype 4h of
Listeria monocytogenes, non-serotype 4h of Listeria monocytogenes,
and Listeria ivanovii.

| | Serotype 4h | Non-serotype 4h | | Listeria |
|---|---|---|---|---|
| | of Listeria | of Listeria | Listeria | monocytogenes + |
| | monocytogenes | monocytogenes | ivanovii | Listeria ivanovii |
| ActA | + | + | − | + |
| SmcL | + | − | + | + |
| I-inIH | − | − | + | + |

Note:
“+” indicates presence,
“−” indicates absence.

### Embodiment 3 Establishment of an Indirect ELISA Method to Determine Whether Sheep is Infected with Listeria monocytogenes and/or Listeria ivanovii

### 1. Determination of Optimal Antigen-Coating Concentration and Optimal Dilution Ratio of Enzyme-Labeled Secondary Antibody.

The optimal antigen-coating concentration and the optimal dilution ratio of the enzyme-labeled secondary antibody were determined by the ELISA checkerboard assay. ActA, SmcL, and I-inIH proteins were diluted to different concentrations (10 μg/mL, 5 μg/mL, 2.5 μg/mL, 1.25 μg/mL, 0.625 μg/mL, 0.31 μg/mL, and 0.16 μg/mL) using carbonate buffer. After dilution, ActA, SmcL, and I-inIH proteins were incubated (100 μL/well) at 4° C. for 16 h, and washed with PBST. The following day, 200 μL of PBS containing 2% BSA was added to each well, incubated at 37° C. for 2 h, and washed with PBST. The same positive and negative goat serum samples, used to determine the optimal reaction conditions, were diluted at a ratio of 1:200 with PBST solution containing 1% BSA. Then, 100.5 μL of the diluted serum samples were added to the wells of the enzyme-linked plate and incubated in a 37° C. water bath for 2 h, followed by washing with PBST. Subsequently, the rabbit anti-goat enzyme-labeled secondary antibody was diluted at a ratio of 1:5000, 1:10000, 1:20000 and 1:40000, respectively. 100 μL of the diluted secondary antibody was added to each well of the enzyme-linked plate and incubated at 37° C. for 1 hour, followed by washing with PBST. TMB was added for color development for 10 minutes, and the reaction was terminated with 2M H₂SO₄ solution. The OD₄₅₀ value was then read with a microplate reader. The optimal antigen-coating concentrations and dilution ratio of rabbit anti-goat enzyme-labeled secondary antibody were selected based on the conditions where the positive OD₄₅₀ value was around 1.0, the negative OD₄₅₀ value was below 0.2, and the positive OD₄₅₀/negative OD₄₅₀ (P/N) ratio was large.

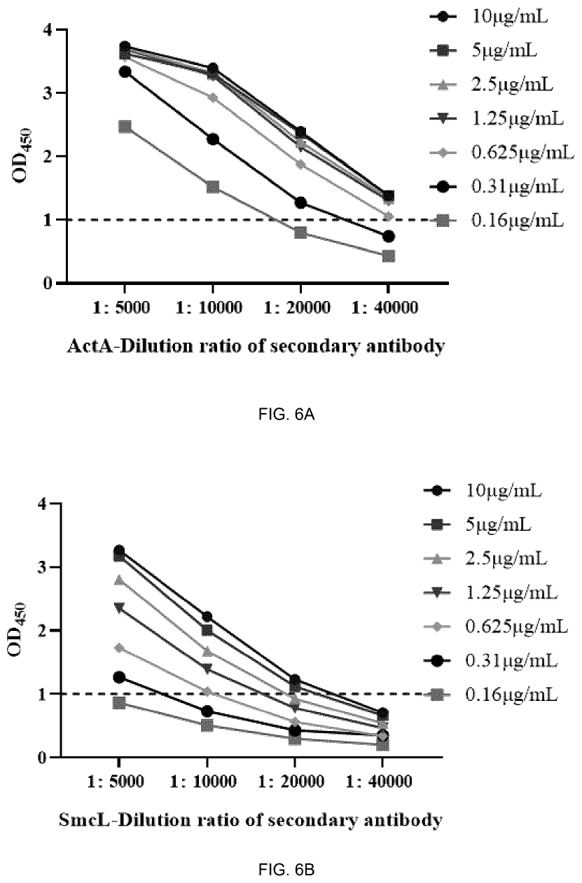

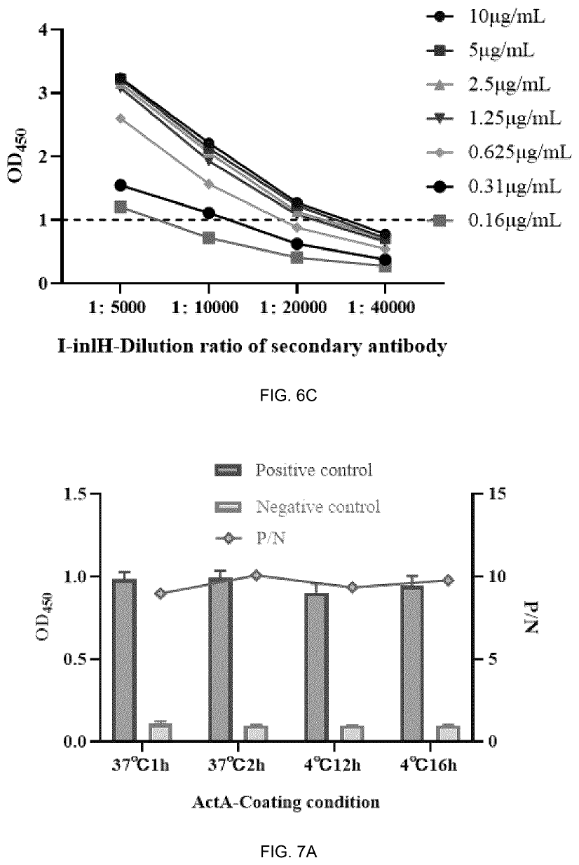

The results are shown in FIG. 6. Considering the cost and usage of the coated-antigen and the enzyme-labeled secondary antibody, the positive OD₄₅₀ value is closest to 1.0 (1.054) when the coating concentration of the ActA antigen is 0.625 μg/mL, and the dilution ratio of the enzyme-labeled secondary antibody is 1:40,000. The positive OD₄₅₀ value is closest to 1.0 (1.037), when the coating concentration of the SmcL antigen is 0.625 μg/mL and the dilution ratio of enzyme-labeled secondary antibody is 1:10,000. The positive OD₄₅₀ value is closest to 1.0 (1.081), when the coating concentration of the I-inIH antigen is 1.25 μg/mL and the dilution ratio of enzyme-labeled secondary antibody is 1:20,000. These conditions provide the most stable indirect ELISA reaction with a high P/N value. Therefore, the optimal antigen-coating concentration and the optimal dilution ratio of enzyme-labeled secondary antibody for the indirect ELISA are determined to be 0.625 μg/mL, 1:40,000 (ActA), 0.625 μg/mL, 1:10,000 (SmcL), and 1.25 μg/mL, 1:20,000 (I-inIH), at which time the concentrations of the secondary antibody are 50 ng/ml, 200 ng/ml, and 100 ng/ml, respectively.

### 2. Determination of Coating Conditions

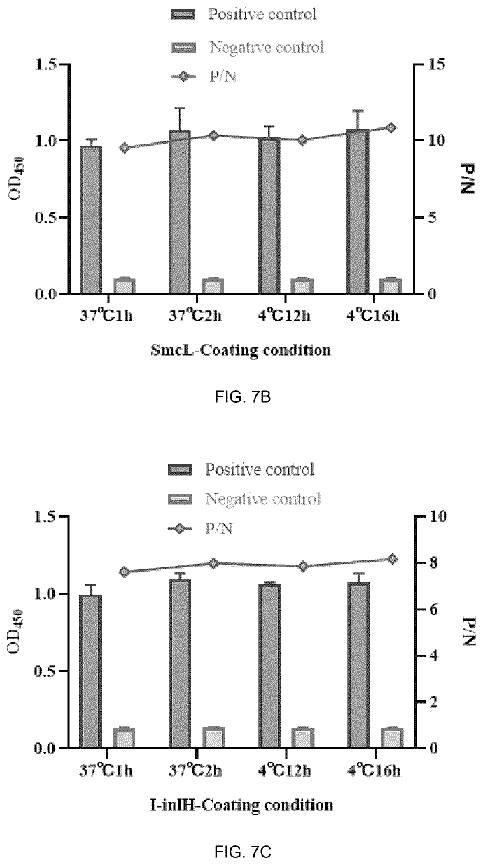

Based on the optimal reaction conditions determined above, four groups of coating conditions were set up to compare the OD₄₅₀ and P/N values of each group. The coating condition with less time and the largest P/N value was selected as the optimal coating condition. As shown in FIG. 7, the optimal coating condition for the ActA in indirect ELISA method is coating at 37° C. for 2 h, and the optimal coating condition for the SmcL and I-inIH in indirect ELISA method is coating at 4° C. for 16 h.

### 3. Determination of Blocking Buffer

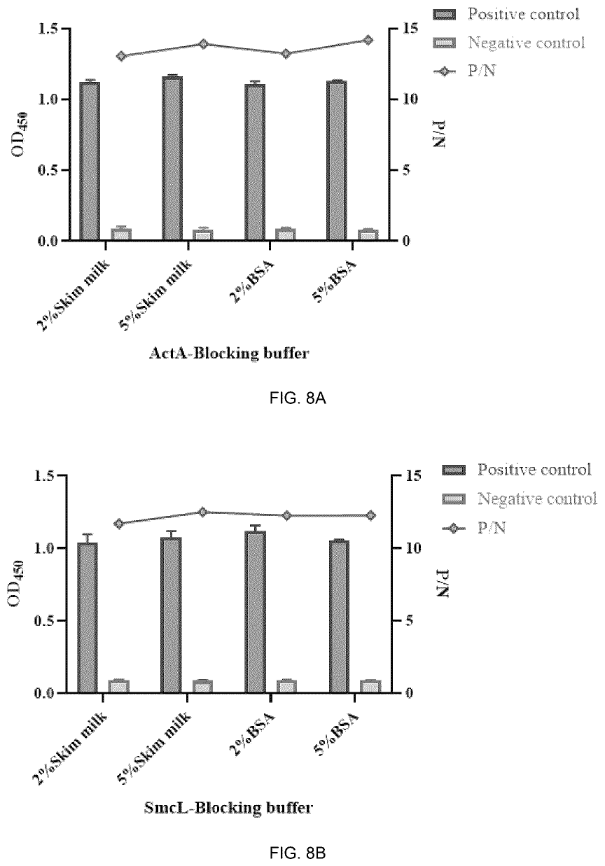

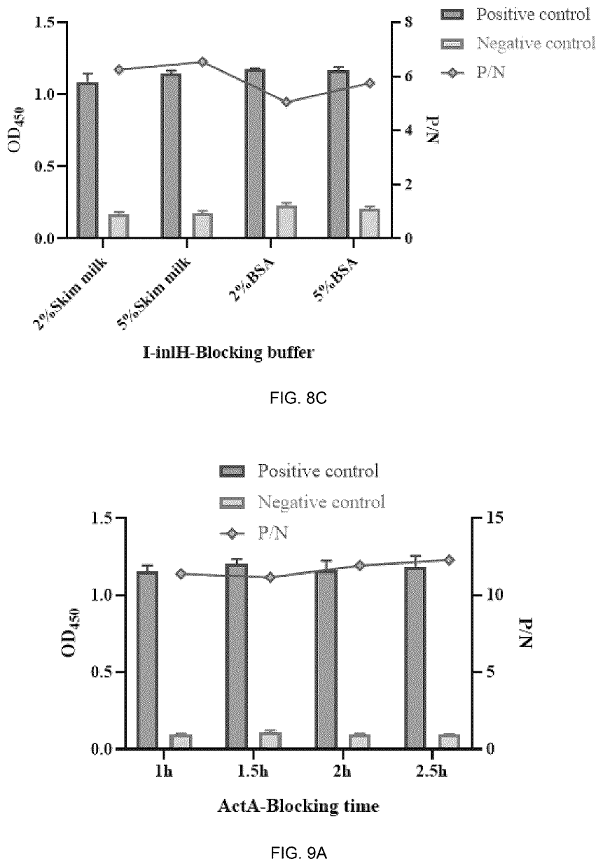

Based on the optimal reaction conditions determined above, four groups of blocking buffers were set up, and the blocking buffer corresponding to the maximum P/N value was selected as the optimal blocking buffer. The results are shown in FIG. 8. PBS containing 5% of BSA is selected as the optimal blocking buffer for the ActA in the indirect ELISA method. PBS containing 5% of skimmed milk is selected as the optimal blocking buffer for the SmcL and I-inIH in indirect ELISA method.

### 4. Determination of Blocking Time

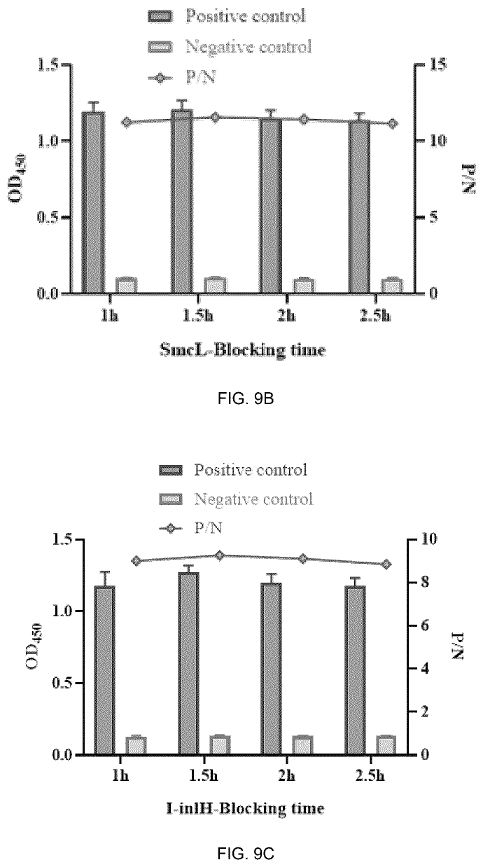

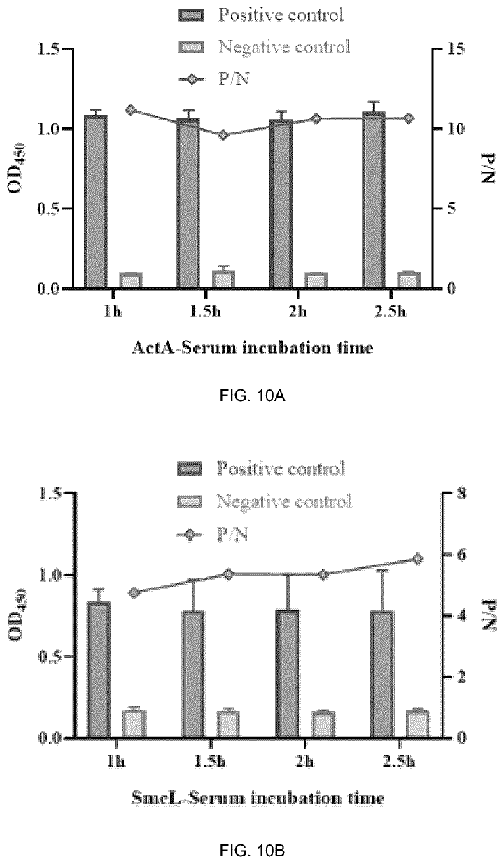

Based on the optimal reaction conditions determined above, four different blocking times were set up, and the condition with the largest average P/N value was selected as the optimal blocking time. The results are shown in FIG. 9, 2.5 h is selected as the optimal blocking time for ActA in indirect ELISA method, and 1.5 h is selected as the optimal blocking time for SmcL and I-inIH in indirect ELISA method.

### 5. Determination of Serum Primary Antibody Incubation Time

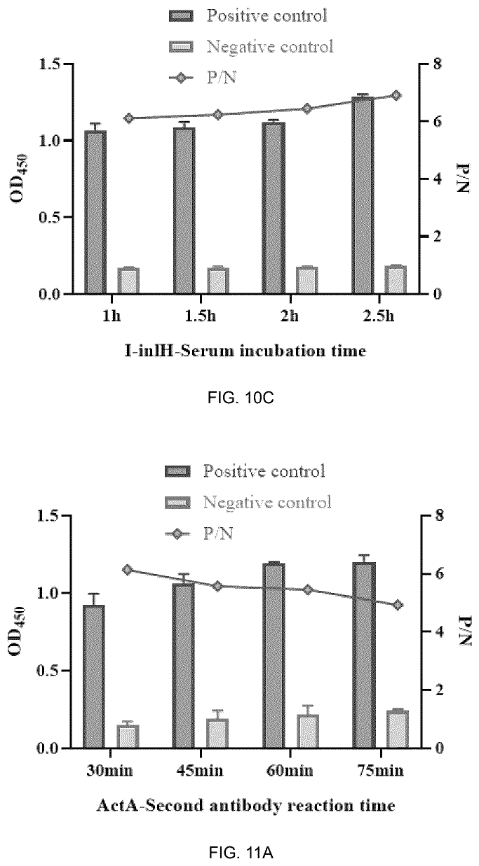

Based on the optimal reaction conditions determined above, positive and negative control serum were diluted according to the dilution ratio of 1:200, and reaction was performed at 37° C. for 1 h, 1.5 h, 2 h, and 2.5 h, respectively. The results are shown in FIG. 10, 1 h is selected as the optimal primary antibody incubation time for the ActA in indirect ELISA method, and 2.5 h is selected as the optimal primary antibody incubation time for the SmcL and I-inIH in indirect ELISA method.

### 6. Determination of Reaction Time of Enzyme-Labeled Secondary Antibody

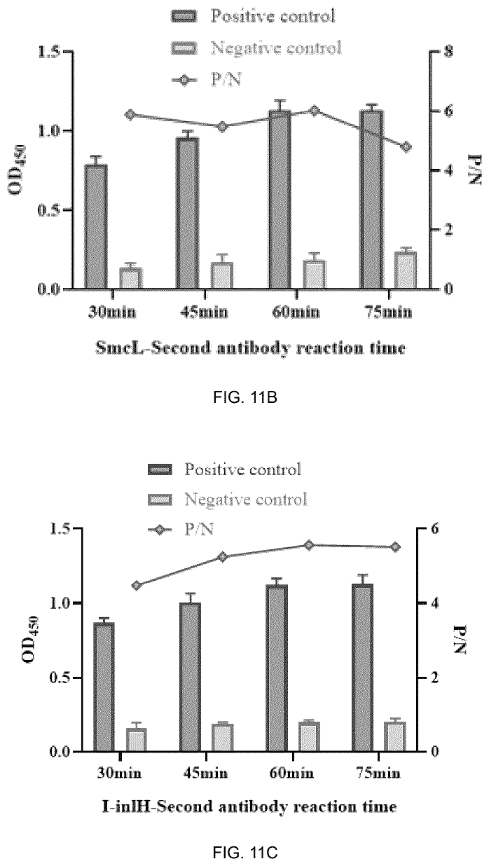

Based on the optimal reaction conditions determined above, different incubation times of HRP-rabbit anti-goat IgG antibody (30 min, 45 min, 60 min, and 75 min) were set up. The results are shown in FIG. 11, 30 min is selected as the optimal secondary antibody reaction time for the ActA in indirect ELISA method, and 60 min is selected as the optimal secondary antibody reaction time for the SmcL and I-inIH in indirect ELISA method.

### 7. Determination of Color-Developing Time

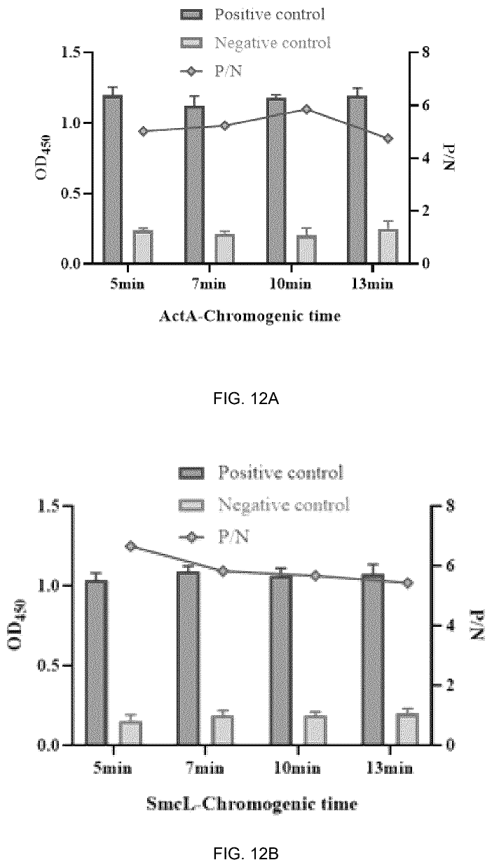

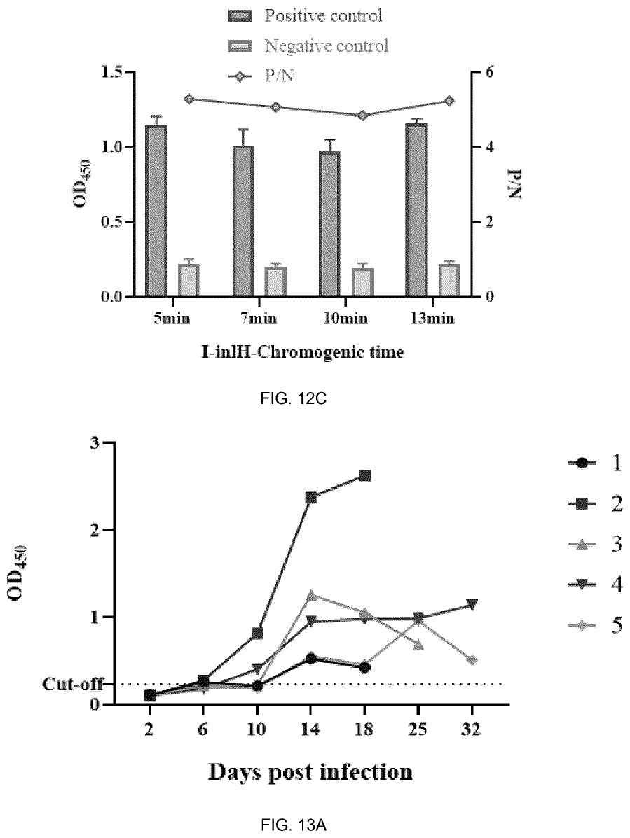

Based on the optimal reaction conditions determined above, four groups of different TMB color-developing times were set up. The results are shown in FIG. 12, 5 min is selected as the optimal color-developing time for the ActA in indirect ELISA method, and 10 min is selected as the optimal color-developing time for the SmcL and I-inIH in indirect ELISA method.

### Embodiment 4 Assembly and Threshold Determination of ELISA Kits for Sheep Listeriosis

The ELISA kit includes 3 antigen-coated plates (consisting of a plate coated with ActA protein, a plate coated with SmcL protein, and a plate coated with I-inIH protein), 800 μL of diluted positive control serum and 800 μL of negative control serum (with a dilution ratio of 1:200), 50 mL of antibody dilution solution (1×), 1 mL of rabbit anti-goat secondary antibody with a dilution ratio of 1:1,000 (which can be further diluted to the optimal ratio with antibody dilution solution when using), 60 mL of PBST washing solution (20×), 30 mL of TMB substrate solution, 20 mL of H₂SO₄ reaction stop solution at 2 mol/L and one user manual.

Thresholds for the ELISA method using ActA, SmcL, and I-inIH as the coated antigens were determined on the basis of the ELISA reaction conditions determined in Embodiment 3. 94 anti-Listeria negative sera that had been confirmed by a third-party were tested, with positive and negative control test values noted as P and N. The mean value x̅ and standard deviation SD of the test results of 94 negative sera were calculated, the critical value L=x̅+3*SD was obtained, then the Cut-off value=(L−N)/(P−N) was further obtained. The OD₄₅₀ value of the tested sample was denoted as S., and the S/P was calculated according to S/P=(S−N)/(P−N), when S/P ratio is greater than the Cut-off value, the serum is positive, and otherwise negative.

The results are shown in Table 2 (a), 2 (b), and 2 (c). For the ActA-iELISA, the detection mean value x̅ is 0.157, the standard deviation SD is 0.024, and the Cut-off value is calculated as 0.104. For the SmcL-iELISA, the detection mean value x̅ is 0.179, the standard deviation SD is 0.018, and the Cut-off value is calculated as 0.110. For the I-inIH-iELISA, the detection mean value x̅ is 0.182, the standard deviation SD is 0.016, and the Cut-off value is calculated as 0.137.

TABLE 2(a)
Results of Threshold of ActA Indirect ELISA

| ActA | 1 | 2 | 3 | 4 | 5 | 6 | 7 | 8 | 9 | 10 | 11 | 12 |
|---|---|---|---|---|---|---|---|---|---|---|---|---|
| A | 0.14 | 0.143 | 0.128 | 0.23 | 0.148 | 0.135 | 0.148 | 0.148 | 0.146 | 0.192 | 0.14 | 0.15 |
| B | 0.132 | 0.137 | 0.224 | 0.135 | 0.15 | 0.135 | 0.227 | 0.222 | 0.14 | 0.137 | 0.135 | 0.135 |
| C | 0.139 | 0.137 | 0.129 | 0.229 | 0.145 | 0.157 | 0.136 | 0.234 | 0.163 | 0.159 | 0.146 | 0.143 |
| D | 0.15 | 0.141 | 0.139 | 0.228 | 0.153 | 0.151 | 0.145 | 0.144 | 0.233 | 0.156 | 0.14 | 0.152 |
| E | 0.138 | 0.145 | 0.138 | 0.143 | 0.148 | 0.153 | 0.141 | 0.151 | 0.151 | 0.151 | 0.156 | 0.165 |
| F | 0.156 | 0.162 | 0.148 | 0.154 | 0.163 | 0.159 | 0.149 | 0.151 | 0.141 | 0.154 | 0.154 | 0.16 |
| G | 0.146 | 0.159 | 0.153 | 0.142 | 0.163 | 0.156 | 0.151 | 0.155 | 0.156 | 0.171 | 0.173 | 0.968 (P) |
| H | 0.163 | 0.171 | 0.153 | 0.155 | 0.18 | 0.168 | 0.151 | 0.156 | 0.159 | 0.168 | 0.161 | 0.143 (N) |

TABLE 2(b)
Results of Threshold of SmcL Indirect ELISA

| SmcL | 1 | 2 | 3 | 4 | 5 | 6 | 7 | 8 | 9 | 10 | 11 | 12 |
|---|---|---|---|---|---|---|---|---|---|---|---|---|
| A | 0.175 | 0.169 | 0.164 | 0.183 | 0.18 | 0.159 | 0.173 | 0.167 | 0.172 | 0.179 | 0.168 | 0.181 |
| B | 0.16 | 0.157 | 0.223 | 0.162 | 0.166 | 0.152 | 0.153 | 0.251 | 0.161 | 0.165 | 0.16 | 0.194 |
| C | 0.164 | 0.159 | 0.155 | 0.165 | 0.167 | 0.158 | 0.163 | 0.176 | 0.162 | 0.221 | 0.164 | 0.175 |
| D | 0.16 | 0.161 | 0.173 | 0.182 | 0.18 | 0.16 | 0.183 | 0.18 | 0.167 | 0.188 | 0.179 | 0.187 |
| E | 0.165 | 0.161 | 0.173 | 0.213 | 0.19 | 0.24 | 0.18 | 0.181 | 0.173 | 0.206 | 0.184 | 0.196 |
| F | 0.199 | 0.163 | 0.171 | 0.19 | 0.18 | 0.179 | 0.197 | 0.185 | 0.169 | 0.184 | 0.179 | 0.199 |
| G | 0.176 | 0.167 | 0.175 | 0.184 | 0.185 | 0.176 | 0.181 | 0.19 | 0.178 | 0.19 | 0.188 | 0.932(P) |
| H | 0.2 | 0.181 | 0.177 | 0.187 | 0.187 | 0.178 | 0.193 | 0.191 | 0.181 | 0.198 | 0.194 | 0.147(N) |

TABLE 2(c)
Results of Threshold of I-inIH Indirect ELISA

| I-inIH | 1 | 2 | 3 | 4 | 5 | 6 | 7 | 8 | 9 | 10 | 11 | 12 |
|---|---|---|---|---|---|---|---|---|---|---|---|---|
| A | 0.17 | 0.172 | 0.168 | 0.189 | 0.17 | 0.172 | 0.169 | 0.17 | 0.173 | 0.174 | 0.179 | 0.193 |
| B | 0.191 | 0.17 | 0.175 | 0.169 | 0.178 | 0.184 | 0.168 | 0.193 | 0.187 | 0.167 | 0.171 | 0.171 |
| C | 0.172 | 0.17 | 0.16 | 0.173 | 0.179 | 0.178 | 0.167 | 0.167 | 0.169 | 0.175 | 0.171 | 0.19 |
| D | 0.179 | 0.178 | 0.174 | 0.179 | 0.209 | 0.186 | 0.175 | 0.183 | 0.185 | 0.172 | 0.195 | 0.198 |
| E | 0.193 | 0.191 | 0.183 | 0.188 | 0.19 | 0.181 | 0.172 | 0.175 | 0.19 | 0.186 | 0.194 | 0.158 |
| F | 0.187 | 0.178 | 0.183 | 0.176 | 0.181 | 0.184 | 0.176 | 0.184 | 0.183 | 0.185 | 0.184 | 0.182 |
| G | 0.19 | 0.183 | 0.178 | 0.273 | 0.178 | 0.186 | 0.187 | 0.18 | 0.187 | 0.187 | 0.194 | 0.866(P) |
| H | 0.215 | 0.187 | 0.228 | 0.191 | 0.194 | 0.193 | 0.194 | 0.159 | 0.2 | 0.188 | 0.191 | 0.129(N) |

### Embodiment 5 Repeatability Tests of ELISA Kit for Sheep Listeriosis

Repeatability tests were performed on the basis of the indirect ELISA method established in Embodiment 3. For the intra-batch repeatability detection, the ELISA plates coated with the purified protein from the same batch were used, and the same six sheep sera were detected using the ELISA plates respectively for three consecutive days. For the inter-batch repeatability detection, the ELISA plates coated with the purified protein from three different batches were used, and the same six sheep sera were detected using the ELISA plates, respectively for three consecutive days. The detection mean value x̅ and standard deviation SD of the samples were analyzed, and finally, the coefficient of variation (CV %) of each sample was calculated by SD/x̅.

The results are as shown in Tables 3 (a), 3 (b), and 3 (c). For the ActA-iELISA, the intra-batch CV ranges from 2.45% to 6.74%, and the inter-batch CV ranges from 3.15% to 8.28%. For the SmcL-iELISA, the intra-batch CV ranges from 1.42% to 5.54%, and the inter-batch CV ranges from 5.25% to 9.61%. For I-inIH-iELISA, the intra-batch CV ranges from 1.96% to 7.23%, and the inter-batch CV ranges from 4.46% to 12.48%. The above results indicate that the ELISA kit has a low degree of variability and good repeatability.

TABLE 3 (a)
Results of ActA-iELISA Repeatability Tests

| | Intra-batch coefficient of | | Inter-batch coefficient of | |
|---|---|---|---|---|
| Serum | variation | | variation | |
| number | x̅ + SD | CV % | x̅ + SD | CV % |
| 1 | 1.208 ± 0.081 | 6.74 | 1.240 ± 0.039 | 3.15 |
| 2 | 0.820 ± 0.043 | 5.27 | 0.781 ± 0.038 | 4.93 |
| 3 | 1.240 ± 0.055 | 4.41 | 1.101 ± 0.091 | 8.28 |
| 4 | 0.195 ± 0.006 | 3.14 | 0.217 ± 0.008 | 3.87 |
| 5 | 0.190 ± 0.005 | 2.45 | 0.215 ± 0.013 | 6.00 |
| 6 | 0.144 ± 0.007 | 4.74 | 0.152 ± 0.009 | 6.02 |

TABLE 3(b)
Results of SmcL-iELISA Repeatability Tests

| | Intra-batch coefficient of | | Inter-batch coefficient of | |
|---|---|---|---|---|
| Serum | variation | | variation | |
| number | x̅ + SD | CV % | x̅ + SD | CV % |
| 1 | 0.919 ± 0.031 | 3.33 | 0.923 ± 0.089 | 9.61 |
| 2 | 1.073 ± 0.015 | 1.42 | 1.131 ± 0.078 | 6.86 |
| 3 | 0.936 ± 0.043 | 4.59 | 0.887 ± 0.059 | 6.64 |
| 4 | 0.170 ± 0.009 | 5.54 | 0.179 ± 0.013 | 7.36 |
| 5 | 0.202 ± 0.007 | 3.26 | 0.216 ± 0.011 | 5.25 |
| 6 | 0.163 ± 0.008 | 4.72 | 0.159 ± 0.014 | 8.99 |

TABLE 3(c)
Results of I-inIH-iELISA Repeatability Tests

| | Intra-batch coefficient of | | Inter-batch coefficient of | |
|---|---|---|---|---|
| Serum | variation | | variation | |
| number | x̅ + SD | CV % | x̅ + SD | CV % |
| 1 | 1.252 ± 0.091 | 7.23 | 1.163 ± 0.145 | 12.48 |
| 2 | 1.538 ± 0.030 | 1.96 | 1.544 ± 0.106 | 6.86 |
| 3 | 0.759 ± 0.036 | 4.77 | 0.885 ± 0.080 | 9.09 |
| 4 | 0.202 ± 0.007 | 3.25 | 0.202 ± 0.009 | 4.46 |
| 5 | 0.146 ± 0.007 | 5.13 | 0.142 ± 0.009 | 6.05 |
| 6 | 0.154 ± 0.004 | 2.93 | 0.153 ± 0.011 | 7.49 |

### Embodiment 6 Utility Test of ELISA Kit for Sheep Listeriosis

### 1. The Dynamic Variation of ActA and SmcL Antibody Levels

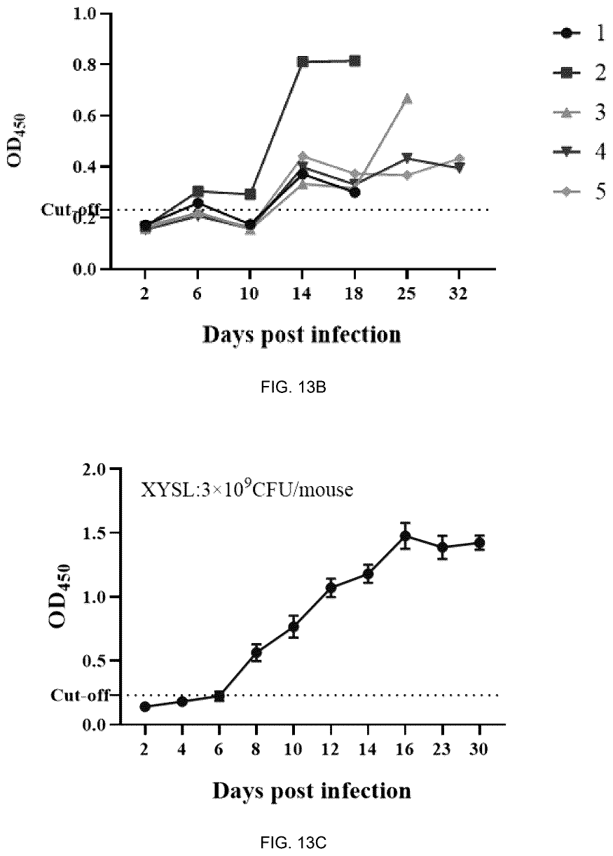

Serotype 4 h of Listeria monocytogenes strain XYSN at a dose of 1.5×10¹⁰ CFU was orally administered to five Hu sheep for infection. ActA and SmcL antibodies in serum were detected according to the indirect ELISA method established in Embodiment 3, and the produced antibody ActA and SmcL in the sheep serum were analyzed. The produced ActA and SmcL antibodies over time in the Hu sheep which have been infected with XYSN are shown in FIGS. 13A and 13B. All Hu sheep are detected as positive for listeriosis by the ActA-iELISA 6 days after infection, that is, they suffer from listeriosis. Around the 18th day, the antibody levels mostly reaches a peak, and the anti-ActA antibodies remain at a relatively high level 32 days after infection. The results indicate that the ActA and SmcL detection components of the kit can diagnose listeriosis both in the early infection stage and for a long time after infection.

### 2. The Dynamic Variation of I-inIH Antibody Levels

Listeria ivanovii strain XYSL at a dose of 3×10⁹ CFU was orally administered to 6-week-old BALB/c mice for infection. I-inIH antibodies in serum were detected according to the indirect ELISA method established in Embodiment 3. The results are shown in FIG. 13C, the BALB/c mice are detected as positive for listeriosis 6 days after infection, that is, they suffer from listeriosis. The antibody level reaches a peak on day 16th, and anti-I-inIH antibodies remain at a relatively high level 30 days after infection. The results indicate that the I-inIH detection components of the kit can diagnose listeriosis in the early infection stage and for a long time after infection.

### Embodiment 7 Thermal Stability Test of the ELISA Kit for Sheep Listeriosis

Thermal disruption of antigen-coated plates included the following steps. The plates were coated and blocked according to the optimum conditions determined in Embodiment 3, then placed in an oven for drying, vacuum-packed in aluminum foil, and placed in a constant temperature incubator at 37° C. for 6, 7, and 8 days, respectively. Thermal disruption of rabbit anti-goat secondary antibody involved the following steps. The rabbit anti-goat secondary antibody was diluted at a ratio of 1:50 using HRP-Protector™ (from Candor Company), sealed, and then placed in a constant temperature incubator at 37° C. for 6, 7, and 8 days, respectively. Thermal disruption of serum controls involved placing diluted positive and negative control serum in a constant temperature incubator at 37° C. for 6, 7, and 8 days, respectively. The intra-batch coefficient of variation of the three sera was tested based on the indirect ELISA method established in Embodiment 3. Positive reference represents positive control, and negative reference represents negative control. The test results are shown in Table 4 (a), 4 (b), and 4 (c). The intra-batch coefficients of variation of all the components in the kit under thermal disruption conditions are lower than 15%, indicating that the thermal stability of the whole kit meets the standard. It is predicted shelf-life of the kit under the condition of 4° C. is more than 8 months.

TABLE 4(a)
Thermal stability analysis of ActA kit

| | | Intra-batch testing indicators | | |
|---|---|---|---|---|
| Thermal | | OD₄₅₀ mean | | |
| disruption time | Reference in Kit | value | SD | CV/% |
| 0 days | Positive reference | 1.115 | 0.111 | 9.96% |
| | Negative reference | 0.130 | 0.009 | 6.92% |
| 6 days | Positive reference | 1.002 | 0.083 | 8.28% |
| | Negative reference | 0.119 | 0.011 | 9.24% |
| 7 days | Positive reference | 0.944 | 0.105 | 11.12% |
| | Negative reference | 0.172 | 0.017 | 9.88% |
| 8 days | Positive reference | 0.907 | 0.090 | 9.92% |
| | Negative reference | 0.169 | 0.017 | 10.06% |

TABLE 4(b)
Thermal stability analysis of SmcL kit

| | | Intra-batch testing indicators | | |
|---|---|---|---|---|
| Thermal | | OD₄₅₀ mean | | |
| disruption time | Reference in Kit | value | SD | CV/% |
| 0 days | Positive reference | 1.252 | 0.119 | 9.50% |
| | Negative reference | 0.139 | 0.012 | 8.63% |
| 6 days | Positive reference | 1.122 | 0.086 | 7.66% |
| | Negative reference | 0.129 | 0.013 | 10.08% |
| 7 days | Positive reference | 0.994 | 0.108 | 10.87% |
| | Negative reference | 0.182 | 0.018 | 9.89% |
| 8 days | Positive reference | 0.987 | 0.098 | 9.93% |
| | Negative reference | 0.179 | 0.025 | 14.53% |

TABLE 4(c)
Thermal stability analysis of the I-inIH kit

| | | Intra-batch testing indicators | | |
|---|---|---|---|---|
| Thermal | | OD₄₅₀ mean | | |
| disruption time | Reference in Kit | value | SD | CV/% |
| 0 days | Positive reference | 1.292 | 0.109 | 8.44% |
| | Negative reference | 0.149 | 0.013 | 8.72% |
| 6 days | Positive reference | 1.222 | 0.096 | 7.86% |
| | Negative reference | 0.128 | 0.012 | 9.38% |
| 7 days | Positive reference | 1.004 | 0.118 | 11.75% |
| | Negative reference | 0.181 | 0.019 | 10.50% |
| 8 days | Positive reference | 0.997 | 0.099 | 9.94% |
| | Negative reference | 0.169 | 0.021 | 12.43% |

### Embodiment 8 Specificity and Sensitivity Test of the ELISA Kit for Sheep Listeriosis

The specificity and sensitivity of the three detection components within the kit were tested on the basis of the indirect ELISA method established in Embodiment 3, and the calculated results are shown in Table 5 (a), 5 (b), and 5 (c). The ActA kit detected 65 sera of sheep infected by Listeria monocytogenes and 92 uninfected sera, and 61 samples were determined to be positive, thus the sensitivity was 93.85%, and 87 samples were determined negative, thus the specificity was 94.57%. The SmcL kit detected 70 sera of sheep infected by serotype 4 h of Listeria monocytogenes or Listeria ivanovii and 92 uninfected sera, and 66 samples were determined positive, thus the sensitivity is 94.29%, and 88 samples were determined negative, thus the specificity is 95.65%. The I-inIH kit detected 15 sera of sheep infected by Listeria ivanovii and 92 uninfected sera, and 15 samples were determined positive, thus the sensitivity is 100%, and 87 samples were determined negative, thus the specificity is 94.57%. The sources of all sera were clear and they had been tested by third-party kits. The results indicate that the detection components within this ELISA kit have good sensitivity and specificity.

TABLE 5(a)
Sensitivity and specificity analysis of ActA kits

| | Infected serum | Uninfected serum |
|---|---|---|
| ActA kit positive | 61 | 5 |
| ActA kit negative | 4 | 87 |
| total | 65 | 92 |
| Sensitivity (%) | 93.85% | / |
| Specificity (%) | / | 94.57% |

TABLE 5(b)
Sensitivity and specificity analysis of SmcL kits

| | Infected serum | Uninfected serum |
|---|---|---|
| SmcL kit positive | 66 | 4 |
| SmcL kit negative | 4 | 88 |
| total | 70 | 92 |
| Sensitivity (%) | 94.29% | / |
| Specificity (%) | / | 95.65% |

TABLE 5(c)
Sensitivity and specificity analysis of the I-inIH kit

| | Infected serum | Uninfected serum |
|---|---|---|
| I-inIH kit positive | 15 | 5 |
| I-inIH kit negative | 0 | 87 |
| total | 15 | 92 |
| Sensitivity (%) | 100% | / |
| Specificity (%) | / | 94.57% |

### Embodiment 9 Compliance Test of ELISA Kit for Sheep Listeriosis

Using the ELISA kit for sheep listeriosis of the present disclosure, some clinical sheep sera with a clear background preserved in a laboratory were detected. The results were compared with those of an imported Listeria Sheep Test Kit (Diatheva) based on the LLO antigen. The results are shown in Table 6 (a), 6 (B), and 6 (c). For the ActA kit, a total of 79 clinical sheep serum samples were tested, and the detection results of 75 samples were consistent with that of the Diatheva kit, indicating a total compliance rate of 94.94%. For the SmcL kit, a total of 79 clinical sheep serum samples were tested, and the detection results of 72 samples were consistent with that of the Diatheva kit, indicating a total compliance rate of 91.14%. For the I-inIH kit, a total of 50 clinical sheep serum samples were tested, and the detection results of 46 samples were consistent with that of the Diatheva kit, indicating a total compliance rate of 92%.

TABLE 6(a)
Comparison between the ActA kit and the Diatheva kit

| | Diatheva kit | | |
|---|---|---|---|
| | Positive | Negative | Total |
| ActA kit positive | 37 | 1 | 38 |
| ActA kit negative | 3 | 38 | 41 |
| Total | 40 | 39 | 79 |

TABLE 6(b)
Comparison between the SmcL kit and the Diatheva kit

| | Diatheva kit | | |
|---|---|---|---|
| | Positive | Negative | Total |
| SmcL kit positive | 38 | 3 | 41 |
| SmcL kit negative | 4 | 34 | 38 |
| Total | 42 | 37 | 79 |

TABLE 6(c)
Comparison between the I-inIH kit and the Diatheva kit

| | Diatheva kit | | |
|---|---|---|---|
| | Positive | Negative | Total |
| I-inIH kit positive | 10 | 1 | 11 |
| I-inIH kit negative | 3 | 36 | 39 |
| Total | 13 | 37 | 50 |

### Embodiment 10 Use of the ELISA Kit for Sheep Listeriosis

Using the indirect ELISA kit of the present disclosure, 40 sheep serum samples from a certain sheep farm in China were collected for detection, and a traceability investigation of the source of the sheep in the sheep farm of Embodiment 10 was conducted. The results showed that three sheep were positive for ActA and SmcL antibodies, indicating that they were infected by serotype 4 h of Listeria monocytogenes, one sheep was positive for ActA antibodies, indicating that it was infected by the non-serotype 4 h of Listeria monocytogenes, twelve sheep were positive for SmcL and I-inIH antibodies, indicating that they were infected by Listeria ivanovii, and fifteen sheep were positive for ActA, SmcL and I-inIH antibodies, indicating that they were infected by Listeria monocytogenes and Listeria ivanovii, with a total antibody positive rate of 77.5%. Moreover, four strains of serotype 4 h of Listeria monocytogenes and one strain of Listeria ivanovii were isolated from wipe samples and environmental samples collected from the sheep farm, suggesting that there is a high positive rate of pathogenic Listeria monocytogenes infections in this sheep farm, and the disinfection and sterilization should be carried out properly.

TABLE 7
Detection results of sheep serum samples from a place
in East China using the ELISA kit for sheep listeriosis

| Type of | | | | | | |
|---|---|---|---|---|---|---|
| infection | A | B | C | D | E | Total |
| 4h Lm | 2 | 0 | 0 | 0 | 1 | 3 |
| Non-4h Lm | 1 | 0 | 0 | 0 | 0 | 1 |
| Liv | 1 | 2 | 2 | 5 | 2 | 12 |
| Lm + Liv | 2 | 4 | 5 | 2 | 2 | 15 |
| Number of | 6 | 6 | 7 | 7 | 5 | 31 |
| positives | | | | | | |
| Total | 8 | 8 | 8 | 8 | 8 | 40 |
| Positive rate | 75% | 75% | 87.5% | 87.5% | 62.5% | 77.5% |

The inventors analyzed the hydrophilicity, surface polarity, antigenicity, and secondary structure characteristics of the I-inIH protein through bioinformatics software, as well as the possible antigenic epitope regions of the protein. The inventors found that the truncated fragment of the I-inIH protein possessed good conservation and immunogenicity. After successfully constructing and expressing the ActA, SmcL and I-inIH proteins, the specific distribution of these three antigens in the serotype 4 h of Listeria monocytogenes, other serotypes of Listeria monocytogenes, and Listeria ivanovii was verified. Subsequently, the inventors developed an indirect ELISA kit for differentiating between Listeria monocytogenes infection and Listeria ivanovii infection. The kit of the present disclosure can effectively differentiate the infections caused by serotype 4 h of Listeria monocytogenes, non-serotype 4 h of Listeria monocytogenes, Listeria ivanovii, or infections of both Listeria monocytogenes and Listeria ivanovii. The kit of the present disclosure has good specificity, sensitivity, and repeatability, facilitating the large-scale detection of listeriosis in sheep farms.

The above are only some preferred embodiments of the present disclosure instead of limitations on the present disclosure in any form or substance. It should be noted that, for those skilled in the art, improvements and supplements may be made without departing from the method of the present disclosure, the improvements and supplements shall also be covered by the present disclosure. The equivalent changes including alternations, modifications, and variations can be made by those skilled in the art using the technical contents revealed above and without departing from the spirit and scope of the present disclosure, and those equivalent changes shall be regarded as equivalent embodiments of the present disclosure. Meanwhile, any alterations, modifications, and variations of any equivalent changes made to the above embodiments according to the essential technology of the present disclosure still fall within the scope of the technical solution of the present disclosure.

1. An indirect ELISA kit for detecting Listeria monocytogenes and/or Listeria ivanovii, comprising:
carriers coating an ActA protein, an SmcL protein, and an I-inIH protein, respectively,
a standard positive control and a standard negative control, and
a detection reagent for detecting an antigen-antibody reaction of the ActA protein, the SmcL protein, and the I-inIH protein with an antibody.

2. The indirect ELISA kit according to claim 1, wherein an amino acid sequence of the ActA protein is shown as SEQ ID NO: 1, an amino acid sequence of the SmcL protein is shown as SEQ ID NO: 2, and an amino acid sequence of the I-inIH protein is shown as SEQ ID NO: 3.

3. The indirect ELISA kit according to claim 1, wherein the antibody comprises a monoclonal antibody or a polyclonal antibody against the ActA protein, a monoclonal antibody or a polyclonal antibody against the SmcL protein, and a monoclonal antibody or a polyclonal antibody against the I-inIH protein;
the detection reagent comprises an enzyme-labeled secondary antibody and a substrate solution;
the marker is selected from a group consisting of horseradish peroxidase (HRP), alkaline phosphatase, and biotin;
the substrate solution is 3,3′,5,5′-tetramethylbenzidine (TMB).

4. The indirect ELISA kit according to claim 1, further comprising a sample dilution solution, a washing solution, a substrate solution, and a reaction stop solution;
the sample dilution solution is a PBST solution containing 1% of BSA,
the washing solution is a PBST solution containing 0.05% of Tween 20 and with a pH of 7.2, and
the reaction stop solution is a 2 mol/L H₂SO₄ solution.

5. The indirect ELISA kit according to claim 1, wherein the standard positive control comprises positive serum from hosts infected with serotype 4 h of Listeria monocytogenes, other strains of Listeria monocytogenes, or Listeria ivanovii;
the standard negative control comprises negative serum from hosts not infected with serotype 4 h of Listeria monocytogenes, other strains of Listeria monocytogenes, or Listeria ivanovii; and
the carriers coated with the ActA protein, the SmcL protein, and the I-inIH protein use ELISA plates.

6. A method for preparing the indirect ELISA kit according to claim 1, comprising the following steps:
(1) coating: coating the ActA protein, the SmcL protein, and the I-inIH protein on ELISA plates, respectively, wherein an amino acid sequence of the ActA protein is shown as SEQ ID NO: 1, an amino acid sequence of the SmcL protein is shown as SEQ ID NO: 2, and an amino acid sequence of the I-inIH protein is shown as SEQ ID NO: 3;
(2) blocking: using a blocking buffer to block the ELISA plates that have been coated with the ActA protein, the SmcL protein, and the I-inIH protein, respectively, as described in step (1);
(3) preparing the standard positive control and the standard negative control;
(4) preparing an enzyme-labeled secondary antibody and a substrate solution;
assembling the ELISA plates prepared in the step (2), the standard positive control and the standard negative control prepared in the step (3), the secondary antibody labeled with the marker and the substrate solution prepared in the step (4) to obtain the indirect ELISA kit.

7. The method according to claim 6, further comprising:
in the step (1), dissolving the ActA protein, the SmcL protein, and the I-inIH protein in a CBS coating solution, respectively, and coating the ActA protein, the SmcL protein, and the I-inIH protein on the ELISA plates at a final concentration of 0.625, 0.625, and 1.25 μg/ml, respectively, wherein the ELISA plate to be coated with the ActA protein is placed at 37° C. for 2 h for the coating, and the ELISA plates to be coated with the SmcL protein and the I-inIH protein are placed at 4° C. for 16 h for the coating;
in the step (2), blocking the ELISA plate coated with the ActA protein with a PBS containing 5% of BSA for 2.5 h, and blocking the ELISA plates coated with the SmcL protein and the I-inIH protein with a PBS containing 5% of skimmed milk for 1.5 h.

8. The method according to claim 5, further comprises preparing a sample dilution solution, a washing solution, a substrate solution, and a reaction stop solution;
wherein the marker is selected from a group consisting of horseradish peroxidase (HRP), alkaline phosphatase, and biotin,
the sample dilution solution is a PBST solution containing 1% w/v of BSA,
the washing solution is a PBST solution containing 0.05% w/v of Tween 20 and with a pH of 7.2,
the substrate solution is 3,3′,5,5′-tetramethylbenzidine (TMB), and
the reaction stop solution is a 2 mol/L H₂SO₄ solution.

9. The method according to claim 5, wherein for the ActA protein, a coating concentration is 0.625 μg/mL, a coating temperature is 37° C., a coating time is 2 h, a blocking buffer is a PBS solution containing 5% of BSA, a blocking time is 2.5 h, and a dilution ration of the secondary antibody labeled with the marker is 1:40,000;
for the SmcL protein, a coating concentration is 0.625 μg/mL, a coating temperature is 4° C., a coating time is 16 h, a blocking buffer is a PBS solution containing 5% of skim milk powder, a blocking time is 1.5 h, and a dilution ration of the secondary antibody labeled with the marker is 1:10,000;
for the I-inIH protein, a coating concentration is 1.25 μg/mL, a coating temperature is 4° C., a coating time is 16 h, a blocking buffer is a PBS solution containing 5% of skim milk powder, a blocking time is 1.5 h, and a dilution ration of the secondary antibody labeled with the marker is 1:20,000.

10. Use of the indirect ELISA kit according to claim 1 in the preparation of a detection reagent for detecting Listeria monocytogenes and/or Listeria ivanovii;
wherein the detection reagent is highly specific for differentiating Listeria monocytogenes and Listeria ivanovii; and
the detection reagent is highly specific for differentiating serotype 4 h of Listeria monocytogenes and other strains of Listeria monocytogenes.

11. A method for detecting Listeria monocytogenes and/or Listeria ivanovii using the indirect ELISA kit according to claim 1, comprising the following steps:
(1) incubation of to-be-tested samples: diluting with a sample dilution solution, followed by incubating the to-be-tested samples on the carriers coating the ActA protein, the SmcL protein, and the I-inIH protein, and adding the corresponding standard positive control and standard negative control;
(2) incubation of a secondary antibody: adding the secondary antibody to the carriers coating the ActA protein, the SmcL protein, and the I-inIH protein after the step (1) and performing incubation;
(3) coloration: adding a detection reagent to the carriers coating the ActA protein, the SmcL protein, and the I-inIH protein after the step (2), performing color development, then stopping the reaction with a reaction stop solution, and reading absorbance values at OD450 nm;
(4) determination of results: calculating S/P ratios, wherein a calculation formula of S/P is S/P=(sample_{absorbance value at OD450 nm}−negative control_{mean absorbance value at OD450 nm})/(positive control_{mean absorbance value at OD450 nm}−negative control_{mean absorbance value at OD450 nm});
for the ActA protein, when a calculated S/P ratio is greater than 0.104, the to-be-tested samples are positive, and otherwise negative;
for the SmcL protein, when a calculated S/P ratio is greater than 0.110, the to-be-tested samples are positive, and otherwise negative;
for the I-inIH protein, when a calculated S/P ratio is greater than 0.137, the to-be-tested samples are positive, and otherwise negative.

12. The method according to claim 11, comprising at least one of the following:
in the step (1), incubating the to-be-tested samples at 37° C. for 1 h on the carrier coating the ActA protein, and incubating the to-be-tested samples at 37° C. for 2.5 h on the carrier coating the SmcL protein and the carrier coating the I-inIH protein, respectively;
the to-be-tested samples in the step (1) are sera;
in the step (2), adding 100 μl of the secondary antibody with a dilution ratio of 1:20,000 to the carrier coating the ActA protein and the carrier coating the SmcL protein after the step (1) and performing incubation for 60 min, and adding 100 μl of the secondary antibody with a dilution ratio of 1:10,000 to the carrier coating the I-inIH protein and performing incubation for 60 min;
in the step (3), a time for the color development for the carrier coating the ActA protein is 5 min, and a time for the color development for the carrier coating the SmcL protein and the carrier coating the I-inIH protein is 10 min.

13. The method according to claim 11, further comprising:
when detection results for the ActA protein, the SmcL protein, and the I-inIH protein all show negative, it indicates that a host do not suffer from listeriosis;
when a detection result for the ActA protein shows positive and detection results for the SmcL protein and the I-inIH protein show negative, it indicates that the host is infected by Listeria monocytogenes rather than serotype 4 h of Listeria monocytogenes;
when detection results for the ActA protein and the SmcL protein show positive and a detection result for the I-inIH protein shows negative, it indicates that the host is infected by serotype 4 h of Listeria monocytogenes;
when a detection result for the ActA protein shows negative and detection results for the SmcL protein and the I-inIH protein show positive, it indicates that the host is infected with Listeria ivanovii;
when detection results for the ActA protein, the SmcL protein, and the I-inIH protein all show positive, it indicates that the host is infected by both Listeria monocytogenes and Listeria ivanovii.

14. Use of an ActA protein, an SmcL protein, and an I-inIH protein in the preparation of an immunological detection reagent for detecting Listeria monocytogenes and/or Listeria ivanovii, wherein the immunological detection reagent further comprises a standard positive control and a standard negative control, and a detection reagent for detecting an antigen-antibody reaction of the ActA protein, the SmcL protein, and the I-inIH protein with an antibody;
the immunological detection reagent is highly specific for differentiating Listeria monocytogenes and Listeria ivanovii; and
the immunological detection reagent is highly specific for differentiating serotype 4 h of Listeria monocytogenes and other strains of Listeria monocytogenes.


SEQUENCE LISTING (12 sequences)

SEQ ID NO: 1 - protein, 610 aa, Listeria monocytogenes
ATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRPTSEEFSSLNSGDFTDDENSETTEEEIDRLADLRDRGTGKHSRNAGFLPLNPFASSPVPSLSPKVSKISAPALISDITKKTPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 2 - protein, 298 aa, Listeria monocytogenes
SDEYPGNFKITSHNVYLFSRNIYPNWGQMHRADLIAQADYMKNNDVVILNEAFDTSASHRLLNNLREMYPHQTPVIGRSKHGWDKTEGNYSNFALEDGGVAVVSQWPIVEKSQHIFQRGGGADRLSNKGFAYVKIMKNGKPYHIIGTHTQADDSLISKDTSRAIRAEQMQEIQTFIAKKNIPKDEIIFIGGDLNVNYGTDEYHDMLKLLNVSSPANFNGQMATWDPTTNSMLKESYPKAAPEYLDYIFVENGHARPHSWHNKVLHTKSPQWSVKSWFKTYTYQDFSDHYPVVGFTDNN

SEQ ID NO: 3 - protein, 285 aa, Listeria ivanovii
ILHSIPINEIFQDYGLANAVKRQLGKQSVTEVVSQRELDEIQKLNGNGCNIKSIKGVEYLTNLTKLYLSYNHISDISALASLSQLEVLSLKQNKLKDINSLANLTKLRCLYVSNNQLRDLWALEGLKNLEYVDAEEQTCTNNPVVYKPKVVIPNTIKDMSGKSVTPNFISDNGEYINTDVIWELPTYTTEVSYRFKDILNIGKDYVLFSGVVIQPVELEAPAAENAPSINQTLPAASLTEALKKGLRKKAVTDISPKSELDKANEFHDENRGSAMFMQLKKSLLM

SEQ ID NO: 4 - DNA, 1833 bp, Listeria monocytogenes
  source 1..1833: mol_type = genomic DNA
gcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagaccaacatctgaagaatttagttcgctgaatagtggtgattttacagatgacgaaaacagcgagacaacagaagaagaaattgatcgcctagctgatttaagagatagaggaacaggaaaacactcaagaaatgcgggttttttaccattaaatccgtttgctagcagcccggttccttcgttaagtccaaaggtatcgaaaataagcgcaccggctctgataagtgacataactaaaaaaacgccatttaagaatccatcacagccattaaatgtgtttaataaaaaaactacaacgaaaacagtgactaaaaaaccaacccctgtaaagaccgcaccaaagctagcagaacttcctgccacaaaaccacaagaaaccgtacttagggaaaataaaacaccctttatagaaaaacaagcagaaacaaacaagcagtcaattaatatgccgagcctaccagtaatccaaaaagaagctacagagagcgataaagaggaaatgaaaccacaaaccgaggaaaaaatggtagaggaaagcgaatcagctaataacgcaaacggaaaaaatcgttctgctggcattgaagaaggaaaactaattgctaaaagtgcagaagacgaaaaagcgaaggaagaaccagggaaccatacgacgttaattcttgcaatgttagctattggcgtgttctctttaggggcgtttatcaaaattattcaattaagaaaaaataattaa

SEQ ID NO: 5 - DNA, 897 bp, Listeria monocytogenes
  source 1..897: mol_type = genomic DNA; serotype = 4h
agtgatgagtatccaggtaattttaagattacttcacacaacgtttatttattttctagaaatatatatcctaattggggacaaatgcaccgagctgatttaattgcacaagcggactatatgaaaaataatgatgtcgtcattttaaacgaagcctttgatacaagtgcttcacatcgactattaaataaccttagagaaatgtatcctcatcaaacacccgtaattgggagaagtaaacatggttgggataaaacggaaggaaattattccaattttgctttggaagatggtggagtggctgtcgttagccagtggcctattgtcgaaaaaagccaacatatttttcagcgcggcggtggcgcagatcggctcagtaataaaggattcgcctatgttaaaattatgaaaaacggaaaaccttatcatataattggaactcatacccaagcggatgactcgcttatttctaaagatactagccgggcgattcgtgcagaacaaatgcaagaaattcaaacatttattgccaagaaaaatatccctaaagatgaaattatctttattggaggcgatttgaacgtaaactacggtacagatgaatatcatgatatgttaaagcttttaaatgttagctctccggcaaactttaacggacagatggctacttgggatccaacgactaattcgatgttaaaagaaagttatcctaaagcagcaccagaataccttgattatatttttgttgaaaacggtcatgcacgtccacattcatggcataacaaagtcttgcataccaagtcaccacagtggtctgtgaaatcttggtttaaaacttatacgtatcaagacttttcagatcattatcctgtagttggtttcactgataataactaa

SEQ ID NO: 6 - DNA, 858 bp, Listeria ivanovii
  source 1..858: mol_type = genomic DNA
attctacattcgatacctataaatgaaatttttcaggattatggtttagcgaatgcagtgaaacgacaattaggaaagcaaagtgttacagaggttgtttcacaaagagaactagatgaaatacaaaagttaaatggtaatggttgcaacattaaatctattaaaggggtagaatatcttactaatttaacaaagttatatctatcttataatcatataagtgatattagcgcgcttgcttctttgagtcaacttgaggtattatctttaaagcagaataagctaaaagacatcaattcattagctaacttgaccaaattaagatgtttgtatgtgagtaacaatcaattaagagatttatgggcattagaagggctgaagaatttagagtatgtagatgcagaagaacaaacctgcaccaataatcctgttgtatacaaaccaaaagtggttattccaaatacgattaaagacatgagcggaaagtcagttacaccaaattttattagcgataatggagaatatataaatacggatgttatatgggagttgccgacatatacaacggaagtaagctatagatttaaggatattctaaacattgggaaagactacgttctatttagtggtgtagtaatacagccagtggaattagaggcacctgctgctgaaaatgcaccctctattaaccaaactttaccagcagcaagtctaacagaagcactaaaaaaaggcttaagaaaaaaggcagttacggatattagtccaaaaagtgaattagataaggcgaatgaatttcacgatgaaaatagaggaagtgcaatgtttatgcaacttaagaaatctctacttatgtga

SEQ ID NO: 7 - DNA, 43 bp, synthetic construct
  source 1..43: mol_type = other DNA
gccatggctgatatcggatccatggcgacagatagcgaagatt

SEQ ID NO: 8 - DNA, 50 bp, synthetic construct
  source 1..50: mol_type = other DNA
gccatggctgatatcggatccatgagtgatgagtatccaggtaattttaa

SEQ ID NO: 9 - DNA, 51 bp, synthetic construct
  source 1..51: mol_type = other DNA
gccatggctgatatcggatccatgattctacattcgatacctataaatgaa

SEQ ID NO: 10 - DNA, 53 bp, synthetic construct
  source 1..53: mol_type = other DNA
ctcgagtgcggccgcaagcttttaattattttttcttaattgaataattttga

SEQ ID NO: 11 - DNA, 50 bp, synthetic construct
  source 1..50: mol_type = other DNA
tcgagtgcggccgcaagcttttagttattatcagtgaaaccaactacagg

SEQ ID NO: 12 - DNA, 48 bp, synthetic construct
  source 1..48: mol_type = other DNA
ctcgagtgcggccgcaagctttcacataagtagagatttcttaagttg